(19) United States
(12) Patent Application Publication
(10) Pub. No.: US 2005/0163764 A1
(43) Pub. Date: Jul. 28, 2005

(54) Treatment with agonists of toll-like receptors

(75) Inventors: Ruslan Medzhitov, Branford, CT (US); Seth Rakoff-Nahoum, New Haven, CT (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Yale University, New Haven, CT (US)

(21) Appl. No.: 10/947,650

(22) Filed: Sep. 22, 2004

Related U.S. Application Data
(60) Provisional application No. 60/587,763, filed on Jul. 13, 2004.
(60) Provisional application No. 60/505,104, filed on Sep. 22, 2003.

(51) Int. Cl. A61K 45/00; A61K 38/16

(52) U.S. Cl. 424/93.45; 514/2

(57) ABSTRACT

Mammals are treated with agonists of bacterially-activated TLRs. The agonist are administered orally or mucosally. In one embodiment, the mammal treated is subject to a gastro-intestinal injury. The agonist can be administered prior to infliction of the gastro-intestinal injury, subsequent to infliction of the gastro-intestinal injury and concurrently with infliction of the gastro-intestinal injury. In another embodiment, the mammal is subject to tissue damage. The agonist is administered prior to the primary treatment, following the primary treatment or concurrently with the primary treatment.

### RELATED APPLICATIONS

This application claims the benefit of U.S. Provisional Application No. 60/587,763, filed Jul. 13, 2004 and 60/505,104, filed Sep. 22, 2003, the entire teachings of both of which are incorporated herein by reference.

### GOVERNMENT SUPPORT

The invention was supported, in whole or in part, by a grant GM07205 from the National Institute of General Medical Sciences and grant AI46688 from the National Institute of Allergy and Infectious Disease. The Government has certain rights in the invention.

### BACKGROUND OF THE INVENTION

Tissue damage can occur in a mammal consequent to treatment of the mammal for a condition, such as a bacterial infection and cancer, or as a result of an injury to a tissue, organ or system of the mammal. The treatments that can cause tissue damage include, for example, antibiotic treatment, chemotherapy, radiation therapy and surgery. Epithelial, connective, nervous and muscle tissue form organs of the mammal that, as a consequence of tissue damage to the mammal, can be functionally compromised, and without repair or protection from further damage can result in death of the mammal. Currently, there are unsatisfactory strategies to prevent, control, manage or repair the tissue damage that can occur as a consequence of certain treatments or injury to tissues, organs or systems of the mammal. Current treatments for mammals undergoing tissue damage, organ damage or some other injury may not effectively activate cellular processes and pathways that prevent tissue damage, mediate tissue repair or prevent further damage. Thus, there is a need to develop new, improved and effective methods of treating mammals that are undergoing or expected to undergo treatments that may result in tissue damage.

### SUMMARY OF THE INVENTION

The present invention relates to methods of treating mammals subject to a gastro-intestinal injury or other tissue damage, such as that consequent to a primary treatment that the mammal is undergoing.

In one embodiment, the method includes treating a mammal, comprising the step of administering an agonist of a bacterially-activated toll-like receptor (TLR) to a mammal subject to a gastro-intestinal injury, wherein the agonist is administered by at least one method of the group consisting of oral administration and mucosal administration and wherein the gastro-intestinal injury is treated.

In another embodiment, the method includes supplementing treatment of a mammal undergoing a primary treatment, wherein the mammal is subject to a tissue damage, comprising the step of administering an agonist of a bacterially-activated TLR to the mammal, wherein the agonist is administered by at least one method of the group consisting of oral administration and mucosal administration.

The invention described herein provides methods of treating mammals subject to a tissue damage or injury to a tissue, organ or system of the mammal. Advantages of the claimed invention include, for example, activation of cellular processes and pathways to prevent tissue damage, mediate tissue repair or prevent further damage to the tissue or organ. Thus, treatment of a mammal with an agonist of a bacterially-activated TLR can prevent, halt, reverse or diminish a gastro-intestinal injury or a tissue damage in a mammal subject to a gastro-intestinal injury or consequent to a primary treatment of the mammal, thereby minimizing complications from certain treatments and tissue damage and decreasing mortality associated with certain treatments.

### BRIEF DESCRIPTION OF THE FIGURES

FIGS. 1A and 1B show increased mortality and morbidity in MyD88−/− Mice following dextran sulfate sodium (DSS) administration. Wild type (WT) control (N=23), knock out mice deficient in MyD88 (MyD88−/−) (N=20), knock out mice deficient in TLR4 (TLR4−/−) (N=18) and knock out mice deficient in TLR2 (TLR2−/−) (N=25) mice received 2% DSS in drinking water for 7 days. On day 8, mice received normal drinking water. Survival was monitored until day 21 after the start of DSS (FIG. 1A). Percent weight change of animals was determined by the following equation: % weight change=(weight at day X−day 0/weight at day 0)×100 (FIG. 1B). Error bars are ±SEM. **=p<0.0=p<0.001 (compared to WT) using the Student's test.

FIGS. 2A, 2B, 2C, 2D and 2E show severe colonic bleeding and anemia in MyD88−/− mice. Kinetics of the severity of colonic bleeding are depicted in FIG. 2A. Colons of WT and MyD88−/− mice were removed at days 0, 1, 3, 5 and 7 post-administration of 2% DSS(N=3-5 mice per timepoint). Scoring was as follows: 0=lack of any gross blood visible throughout the entire colon; 1=gross blood present in <⅓ of the colon; 2=<⅔; 3=>⅔ of the colon. UD=undetected. FIGS. 2A, 2B and 2C show photomicrographs of a representative colon from WT and MyD88−/− mice at day 5 of DSS-treatment. RBC concentration (FIG. 2D) and hematocrit values (FIG. 2E) of peripheral blood taken at various timepoints during the administration of 2% DSS for 7 days are shown. Error bars represent ±SEM. *=p<0.05, **=p<0.01, =p<0.001 (compared to WT) using the Student's test.

FIGS. 3A, 3B, 3C, 3D, 3E, 3F and 3G show colonic epithelial damage in MyD88−/− mice following DSS administration. FIG. 3A-3D show representative photomicrographs (magnification, ×200; hematoxylin and eosin staining) of colons from WT and MyD88−/− mice at days 0 and 5 of DSS administration. Histopathological scoring (FIG. 3E), ulcer and erosions (FIG. 3F), epithelial injury and infiltrating leukocytes (FIG. 3G) of colons from WT and MyD88−/− mice at days 0, 3 and 5 of DSS administration are shown. UD=undetected. Error bars represent ±SEM. *=p<0.05 (compared to WT) using the Student's test.

FIG. 4A, 4B, 4C, 4D, 4E, 4F, 4G, 4H, 4I, 4J, 4K, 4L, 4M, 4N and 4O show defects in steady-state intestinal epithelial homeostasis in the absence of TLR signaling. FIGS. 4A-4H show photomicrographs of immunhistochemical staining for BrDU from sections of colons of WT and MyD88−/− mice injected with 1 mg/ml BrDU and sacrificed 24 hours (upper panels) and 2 hours (lower panels) later. Sections were counterstained with hematoxylin. Magnifications: For 24 h: upper panel, ×200; lower panel, ×400; For 2 h: upper panel, ×100; lower panel, ×400. FIG. 4M shows the average number of cells per one side of colonic crypt of WT and MyD88−/− mice. FIG. 4N shows the protein lysates isolated from colonic epithelium of WT and MyD88−/− were analyzed by western blot for cyclin D1 and β-actin. FIGS. 4I-4L show the photomicrographs of 2 hour BrDU staining of WT and MyD88−/− colons 3.5 days after 10 Gy whole body irradiation; upper panel ×40, lower panel ×100. FIG. 4O shows the average number of BrDU+ cells per crypt at 2 hours post injection of BrDU at baseline (day 0) and 3.5 days after 10 Gy whole body irradiation. Error bars represent ±SEM. **=p<0.01, (compared to WT) using the Student's test.

FIGS. 5A, 5B, 5C, 5D, 5E and 5F show MyD88 dependent induction of cytokines in the colon by commensals. FIGS. 5A-5C show the baseline endogenous production of IL-6, KC-1 and TNF by the colons of uninjured WT, MyD88−/− and WT mice depleted of commensals by 4 week administration of broad-spectrum antibiotics (WT+Abx); UD=undetected. FIGS. 5D and 5E show the induction of IL-6 and KC-1 in WT and MyD88−/− colons at 3, 5, 7 and 9 days after the initiation of DSS. Fold induction was determined by dividing the concentration of factor at each timepoint by the value at day 0. Data is representative of 2-3 experiments per time-point. Factors are derived from spontaneous release into supernatant after 24 hour whole organ culture of colons in serum free media. Cytokines in the supernatant were measured by ELISA and were normalized for the amount of cytokine per mg of total protein in supernatant. FIG. 5F shows the protein lysates isolated from colonic epithelium of WT and MyD88−/− mice were analyzed by western blot for Hsp25, Hsp72 and β-actin. Error bars represent ±SEM. *=p<0.05 (compared to WT) using the Student's test.

FIGS. 6A and 6B show depletion of colonic microflora by broad-spectrum antibiotics. Animals were given ampicillin (A; 1 g/L), vancomycin (V; 500 mg/L), neomycin sulfate (N; 1 g/L) and/or metronidazole (M; 1 g/L) in drinking water for 4 weeks prior to beginning DSS treatment (FIG. 6A). Three depletion protocols were used: A/V/N/M, V/M and N/M. After 4 weeks on antibiotics, colonic fecal matter was cultured aerobically and anaerobically and commensal bacteria were identified and quantified using biochemical analysis, morphologic appearance and Gram staining. Survival of animals treated with the above combinations of antibiotics for 4 weeks upon administration of 2% DSS in drinking water for 7 days (FIG. 6B).

FIGS. 7A, 7B, 7C, 7D, 7E and 7F show protection from gut injury is dependent on recognition of commensal derived ligands by TLRs. Survival (FIG. 7A) and percent weight change (FIG. 7B) of WT animals depleted of commensals by a 4 week regimen of A/V/N/M (Comm. depl.+DSS), commensal-depleted animals reconstituted with either 50 μg/μl of purified E. coli 026:B6 LPS (Comm. depl.+DSS+LPS) or 12.5 μg/μl S. aureus lipoteichoic acid (LTA) (Comm. depl.+DSS+LTA), and undepleted mice (DSS) after 7 day administration of 2% DSS are shown. LPS and LTA were administered in drinking water for the week prior to and during DSS exposure. FIG. 7C shows a photomicrograph of representative colons from commensal-depleted WT mice with and without oral reconstitution of LPS at day 5 of DSS-treatment. Protein lysates isolated from colonic epithelium of WT animals, without antibiotics (undepleted) commensal depleted, and commensal depleted and reconstituted with oral LPS, were analyzed by western blot for Hsp25, Hsp72 and β-actin (FIG. 7D). FIGS. 7E and 7F show survival of TLR4−/− and TLR2−/− depleted of commensals by a 4 week regimen of A/V/N/M (Comm. depl.+DSS), commensal-depleted animals reconstituted with 50 μg/μl of purified E. coli 026:B6 LPS (Comm. depl.+DSS+LPS), and undepleted mice (DSS) after 7 day administration of 2% DSS. Error bars represent ±SEM. **=p<0.01 (Commensal depleted+DSS+LPS and +LTA compared to Commensal depleted+DS) using the Student's test.

FIG. 8 shows the number or character of infiltrating leukocytes per unit area of intestine. The average number of polymorphonuclear cells (PMN), lymphocytes (Ly), eosinophils (Eos), and total leukocytes (Total) per high power field (×400) of WT and MyD88−/− colons at day 5 post-DSS is depicted. Error bars represent ±SEM.

FIG. 9 shows survival of WT and MyD99−/− mice. Mice deficient in TLR signaling are more susceptible to radiation-induced mortality. WT and MyD88−/− mice were exposed to 10 Gy of gamma irradiation at 1.9 Gy/min and followed for survival. Mice were reconstituted with 3×10⁶ bone marrow cells and placed on prophylactic antibiotics to control for mortality due to radiation-induced bone marrow depletion.

FIG. 10 shows LPS rescue of DSS induced mortality upon commensal depletion is dose-dependent. Survival of WT animals depleted of commensals by a 4 week regimen of A/V/N/M (Comm. depleted+DSS), commensal-depleted animals reconstituted with either 10 μg/μl of purified E. coli 026:B6 LPS (Comm. depleted+DSS+10 μg/μl LPS) or 10 ng/μl LPS (Comm. depleted+DSS+10 ng/μl LPS), and undepleted mice (DSS) after 7 day administration of 2% DSS. LPS was administered in drinking water for the week prior to and during DSS exposure.

FIG. 11 depicts the wound area in wild type (WT) mice, mice deficient in TLR2 and TLR4 (TLR2/4−/−) and knock out MyD88 (MyD88−/−) mice in days following the infliction of a wound.

### DETAILED DESCRIPTION OF THE INVENTION

The features and other details of the invention, either as steps of the invention or as a combination of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

The present invention relates to methods of treating mammals subject to a gastro-intestinal injury or other tissue or organ damage, such as that consequent to a primary treatment that the mammal is undergoing. It has been discovered that activation of bacterially-activated TLRs can prevent tissue or organ damage, prevent continued damage to the tissue or the organ that is damaged and promote tissue or organ repair in a damaged tissue or organ.

In one embodiment, the method includes treating a mammal, comprising the step of administering an agonist of a bacterially-activated TLR to a mammal subject to a gastro-intestinal injury, wherein the agonist is administered by at least one method of the group consisting of oral administration and mucosal administration and wherein the gastro-intestinal injury is treated.

Toll-like receptors (TLRs) are type I transmembrane proteins know to be involved in innate immunity by recognizing microbial conserved structures. TLRs may also recognize endogenous ligands induced during the inflammatory response. There are eleven TLRs (TLR1, TLR2, TLR3, TLR4, TLR5, TLR6, TLR7, TLR8, TLR9, TLR10 and TLR11) (Janeway, C. A., Jr., et al., Annu Rev Immunol 20: 197-216 (2002) and Zhang, D., et al., Science 303: 1522-1526 (2004)) that differ in the microbial product that activates the TLR. For example, TLR1, TLR2, TLR4, TLR5 and TLR6 recognize or is activated by bacterial products (e.g., Gram positive and Gram negative bacteria). TLR3, TLR7 and TLR8 recognizes viral products (e.g., dsRNA, viral RNA). TLR9 recognizes bacterial and viral products (e.g., unmethylated CpG motifs frequently found in the genome of bacteria and viruses, but not vertebrates). TLR2 also recognizes fungal, such as yeast, products (e.g., zymoson, mannan). Plasmacytoid dendritic cells express TLR3, TLR7 and TLR9.

“Bacterially-activated TLR,” as used herein, refers to a toll-like receptor (TLR) that recognizes bacterial structures. The bacterial structure can be any portion or fragment of a bacteria (e.g., Gram negative or Gram positive bacteria) that, upon recognition by the TLR by, for example, binding to the extracellular domain of the TLR, results in activation of the TLR to mediate cellular processes. In one embodiment, the bacterially-activated TLR is not a TLR9.

“Virally-activated TLR,” as used herein, refers to a TLR that recognizes viral structures. The viral structure can be any portion or fragment of a virus (e.g., double-stranded RNA and viral RNA) that, upon recognition by the TLR by, for example, binding to the extracellular domain of the TLR, results in activation of the TLR to mediate cellular processes.

“Bacterially- and virally-activated TLR,” as used herein, refers to a TLR that recognizes bacterial and viral structures. The bacterial structure recognized by a bacterially- and virally-activated TLR can be any portion or fragment of a bacteria (e.g., Gram negative or Gram positive bacteria) that, upon recognition by the TLR by, for example, binding to the extracellular domain of the TLR, results in activation of the TLR to mediate cellular processes. The viral structure recognized by a bacterially- and virally-activated TLR can be any portion of a virus, for example, double-stranded RNA and viral RNA that, upon recognition by the TLR by, for example, binding to the extracellular domain of the TLR, results in activation of the TLR to mediate cellular processes.

“Fungally-activated TLR,” as used herein, refers to a TLR that recognizes fungal (e.g., yeast) structures. The fungal structure recognized by a fungally-activated TLR can be any portion or fragment of a fungus (e.g., yeast) that, upon recognition by the TLR by, for example, binding to the extracellular domain of the TLR, results in activation of the TLR to mediate cellular processes. The fungal structure recognized by a fungally-activated TLR can be yeast or any portion of a yeast, for example, zymoson and mannan.

Because the cytoplasmic domain of many TLRs is highly conserved, they employ similar signaling molecules, such as interleukin 1 receptors (IL-1Rs) which include MyD88, IL-1R-associated protein kinase and tumor necrosis factor receptor-activated factor 6 to mediate responses to activation. The bacterially-activated TLR employed in the methods of the invention can stimulate activation of IL-1Rs, including MyD88, IL-1R-associated protein kinase and tumor necrosis factor receptor-activated factor 6.

The term “agonist,” as used herein, refers to an agent that activates cell signaling of a TLR, such as a bacterially-activated TLR, a virally activated TLR, a bacterially- and virally-activated TLR and a fungally-activated TLR, with the proviso that the agonist is not a commensal bacteria.

The agonist can be a naturally occurring activator of a TLR, such as LPS, a ligand for TLR4; flagellin, a ligand of TLR5; double-stranded RNA, a ligand for TLR3; and viral RNA, a ligand for TLR7. The agonist can also be a synthetic activator for a TLR, such as an LPS-mimetic (Corixa Corporation, Seattle, Wash.) that activates TLR4; and imiquimode that activates TLR7.

The agonist can activate cell signaling of a bacterially-activated TLR by, for example, interacting with the TLR (e.g., binding the TLR) or activating any downstream cellular pathway that occurs upon binding of a ligand to a TLR. An agonist of a bacterially-activated TLR can also enhance the availability or accessability of an endogenous or naturally occurring ligand of the TLR. The agonist of the bacterially-activated TLRs can alter transcription of genes, increase translation of mRNA or increase the activity of proteins that are involved in mediating TLR cellular processes. For example, the agonists of bacterially-activated TLRs can increase TNF, IL-6 and KC-1.

In one embodiment, a second agonist of the bacterially-activated TLR can be a bacteria or any fragment or portion of bacteria that activates cell signaling through a bacterially-activated TLR. “A second agonist,” as used herein, can be at least one component of a primary treatment. For example, the bacteria can be commensal bacteria or a fragment thereof. “A fragment or portion of a bacteria,” as used herein, refers to any part of the bacteria that activates cell signaling through a bacterially-activated TLR. Commensal bacteria can be found in the gastro-intestinal tract of a mammal and activate TLRs, including TLR2 and TLR4. “Gastro-intestinal commensal bacteria,” as used herein, refers to commensal bacteria in the gastro-intestinal tract (e.g., mouth, tongue, pharynx, esophagus, stomach, duodenum, jejunum, ileum, cecum, colon, rectum, anal canal) of the mammal. Commensal bacteria activate TLRs is a non-sterile environment. Agonists employed in the methods of the invention can also activate TLRs in aseptic environments.

In another embodiment, the agonist employed in the methods of the invention activates at least one member selected from the group consisting of a TLR1, TLR2, TLR4, TRL6 and TLR5. In yet another embodiment, the agonist activates at least one member selected from the group consisting TLR3, TLR7 and TLR8, wherein the agonist is administered by at least one method selected from the group consisting of an intramuscular, an intradermal and an intravenous administration. The agonist of TLR2 can be at least one member selected from the group consisting of a lipoteichoic acid, a peptidoglycan, lipoprotein and outer-surface lipoprotein (OspA). The agonist of TLR7 can be at least one member selected from the group consisting of a viral RNA and imiquimode. The agonist of TLR3 can be double-stranded RNA. The agonist of TLR2 can be zymoson and mannan.

The agonist of TLR4 can be a lipopolysaccharide, such as a lipopolysaccharide of Salmonella minnesota R595 (e.g., monophosphoryl lipid A).

The mammal treated by the method of the invention can be, for example, a human, mouse, rat or monkey.

The mammal to be treated by the methods of the invention is subject to a gastro-intestinal injury. A “gastro-intestinal injury,” as used herein, refers to any disruption of the homeostasis of any tissue (epithelial, connective, nervous or muscle) of any organ or compartment of the gastrointestinal tract of the mammal. The gastro-intestinal injury can be a consequence of an endogenous disruption of the homeostatis of any tissue of the gastrointestinal tract, such as a cancer. Alternatively, or additionally, the gastro-intestinal injury can be a consequence of an exogenous disruption of the homeostasis of any tissue of the gastrointestinal tract, for example, injury consequent to at least one member selected from the group consisting of antibiotic treatment, surgery, chemotherapy and radiation therapy, or some external impact causing injury to the mammal. “The gastro-intestinal injury is treated,” as used herein, refers to treatment or prevention of the gastro-intestinal injury.

In another embodiment, the mammal to be treated by the methods of the invention is subject to a tissue injury or an organ injury, such as an injury in an epithelial tissue, connective tissue, muscle tissue or neuronal tissue or an injury in at least one organ selected from the group consisting of the skin, heart, liver, kidney, pancreas, spleen, bone, bone marrow, pharynx and larynx. “A tissue injury” or “an organ injury,” as used herein, refers to any disruption of the homeostasis of any tissue (epithelial, connective, nervous or muscle) of any organ of the mammal. The tissue or organ injury can be a consequence of an endogenous disruption of the homeostatis of any tissue or any organ, such as a cancer. Alternatively, or additionally, the organ or tissue injury can be a consequence of an exogenous disruption of the homeostasis of any organ or any tissue, for example, injury consequent to at least one member selected from the group consisting of antibiotic treatment, surgery, chemotherapy and radiation therapy, or some external impact causing injury to the mammal.

In one embodiment, the epithelial of an organ or the gastrointestinal tract of the mammal is injured. The epithelium that is injured can be stratified squamous epithelial, for example of the esophagus, or simple columnar epithelium of the stomach, large intestine and small intestine or any other organ of the mammal, such as a hepatocyte. In a preferred embodiment, the epithelium is a mucosal epithelium of, for example, the gastro-intestinal tract. The integrity of the basement membrane of the epithelium can be compromised as a result or consequent to the organ or tissue injury, for example, a gastrointestinal injury, an injury to the liver or an injury to the skin. The basement membrane can be partially or completing compromised in the injury. Disruptions in the integrity of the basement membrane can significantly compromise the ability of the organ, tissue, or, for example, the gastro-intestinal tract or liver to function, resulting in hemorrhage.

In another embodiment, the organ or tissue injury (e.g., a gastro-intestinal injury, injury to the bone marrow, injury to the liver, injury to the skin) can be to connective tissue (e.g., stroma, fibroblasts, extracellular matrix), the muscle (e.g., smooth muscle, skeletal muscle, cardiac muscle) and/or neurons of the organ or tissue (e.g., the gastrointestinal tract, liver, bone).

The segment of the gastrointestinal tract that is injured is at least one member selected from the group consisting of the mouth, tongue, pharynx, esophagus, stomach, small intestine (duodenum, jejunum and ileum) and large intestine (cecum, colon, rectum and anal canal). One of skill in the art would be able to employ routine diagnostic criteria to identify a mammal with a tissue or organ injury, such as a gastro-intestinal injury, and the type and extent of the injury.

The gastro-intestinal injury can be, for example, polyposis. In one embodiment, the polyposis is familial intestinal polyposis. In another embodiment, the polyposis is multiple intestinal polyposis.

In another embodiment, the gastro-intestinal injury can be regional enteritis (Crohn's disease).

In still another embodiment, the injury is of the large intestine injury and is at least one member selected from the group consisting of colon cancer and ulcerative colitis.

In one embodiment, the agonists of the bacterially-activated TLR is administered prior to infliction of the gastrointestinal injury. “Prior to infliction of gastro-intestinal injury,” as used herein, refers to any point in time before the mammal has a gastro-intestinal injury. For example, the agonist can be administered to the mammal before the mammal is scheduled to undergo a procedure or treatment that typically results in gastro-intestinal injury. Administration of the agonist prior to infliction of the gastro-intestinal injury can prevent or minimize the gastro-intestinal injury that results. For example, the mammal can be administered the agonist before undergoing a scheduled regimen of antibiotic, chemotherapy, radiation therapy or surgical treatment, which inflict gastro-intestinal injury to the mammal.

The agonists of the bacterially-activated TLR is administered prior to infliction of a tissue or organ injury in the mammal. “Prior to infliction of tissue or organ injury,” as used herein, refers to any point in time before the mammal has a tissue or organ injury. For example, the agonist can be administered to the mammal before the mammal is scheduled to undergoing a procedure or treatment that typically results in a tissue or organ injury. Administration of the agonist prior to infliction of the tissue or organ injury can prevent or minimize the tissue or organ injury that results. For example, the mammal can be administered the agonist before undergoing a scheduled regimen of antibiotic, chemotherapy, radiation therapy or surgical treatment, which inflict tissue or organ injury to the mammal.

In another embodiment, the agonist of the bacterially-activated TLR is administered to the mammal subsequent to the infliction of the gastro-intestinal injury. “Subsequent to the infliction of the gastrointestinal injury,” as used herein, refers to the administration of the agonist after the gastro-intestinal injury is present in the mammal. For example, the mammal can have gastro-intestinal injury subsequent to the administration of antibiotics, chemotherapy, radiation therapy or surgery. The agonist is then administered to the mammal to treat the gastro-intestinal injury.

In another embodiment, the agonist of the bacterially-activated TLR is administered to the mammal subsequent to the infliction of the tissue or organ injury. “Subsequent to the infliction of the tissue or organ injury,” as used herein, refers to the administration of the agonist after the tissue or organ injury is present in the mammal. For example, the mammal can have a tissue or organ injury subsequent to the administration of antibiotics, chemotherapy, radiation therapy or surgery. The agonist is then administered to the mammal to treat the tissue or organ injury.

In yet another embodiment, the agonist of the bacterially-activated TLR is administered to the mammal concurrently with infliction of the gastro-intestinal injury. “Concurrently with infliction of the gastro-intestinal injury,” as used herein, refers to the administration of the agonist simultaneously with the treatment or procedure that results in the gastro-intestinal injury. Administration of the agonist concurrent with infliction of the gastro-intestinal injury can be administration of the agonist and the treatment or procedure that results in gastro-intestinal injury at about the same point in time. For example, the agonist can be co-administered to the mammal with a treatment or procedure that results in gastro-intestinal injury.

In yet another embodiment, the agonist of the bacterially-activated TLR is administered to the mammal concurrently with infliction of the tissue or organ injury. “Concurrently with infliction of the tissue or organ injury,” as used herein, refers to the administration of the agonist simultaneously with the treatment or procedure that results in the tissue or organ injury. Administration of the agonist concurrent with infliction of the tissue or organ injury can be administration of the agonist and the treatment or procedure that results in tissue or organ injury at about the same point in time. For example, the agonist can be co-administered to the mammal with a treatment or procedure that results in tissue or organ injury.

Co-administration is meant to include simultaneous or sequential administration of the agonist and treatment or procedure that results in the tissue or organ (e.g., gastro-intestinal injury, bone marrow injury, liver injury or skin injury), individually or together. Where the agonist is concurrently administered to the mammal with infliction of the tissue or organ injury (e.g., gastro-intestinal injury, bone marrow injury, liver injury or skin injury) it is preferred that the administration of the agonist is conducted sufficiently close in time to treatment or procedure. For example, administration of the agonist is sufficiently close in time to administration of, for example, a chemotherapeutic agent, radiation treatment, surgery, ingestion of an antibiotic, so that the effects of the treatment or procedure on tissue or organ injury (e.g., gastro-intestinal injury, bone marrow injury, liver injury or skin injury) are absent or minimized.

In another embodiment, the invention is a method for supplementing treatment of a mammal undergoing a primary treatment. The primary treatment may be one that causes damage to a tissue or an organ that can be prevented or alleviated by administering an agonist of a bacterially-activated TLR to the mammal. In one embodiment, the agonist is administered by at least one method of the group consisting of oral administration and mucosal administration. The primary treatment, in one embodiment, can be the cause of the injury. The injury is any of those discussed above, such as gastro-intestinal injury.

“Supplementing treatment of a mammal undergoing a primary treatment,” as used herein, refers to the addition of the administration of an agonist of a bacterially-activated TLR to a treatment regimen, the primary treatment, for a condition or disease in the mammal.

“Primary treatment,” as used herein, refers to a remedy, medication, procedure or technique prescribed or designed for a particular condition. The primary treatment can be at least one member selected from the group consisting of chemotherapy and radiation therapy for a cancer or other condition or disease for which it is desirous to administer chemotherapy or radiation therapy.

The primary therapy can be surgery (e.g., abdominal surgery, thoracic surgery, pelvic surgery, oral surgery, orthopedic). The surgery can accompany or occur in a sequence of time with another primary procedure, such as a bone marrow transplant, chemotherapy or radiation therapy.

The mammal can be undergoing one or more primary treatments either sequentially or in combination when the primary treatment is supplemented with the agonist of the bacterially-activated TLR. In an embodiment, the agonist is administered to a mammal undergoing a bone marrow transplant, radiation treatment and chemotherapy.

The primary therapy can be antibiotic treatment. The antibiotics used as a primary therapy in a mammal or that result in gastro-intestinal injury can be, for example, metronidazole or quinilones (e.g., ciprofloxin), which can be used as a primary treatment for fistulizing and colonic involvement in Crohn's Disease (Sutherland L, et al., Gut 32: 1071-1075 (1991) and Podolsky D. K., New Engl. J. Med. 347: 417-429 (2002), the teachings of both of which are hereby incorporated by reference in their entirety). The antibiotics can also be cephalosporins, such as cephalexin and ceftriaxone.

The antibiotic can be used in combination with another primary treatment. For example, cephalosporins can be used as prophylactic therapy for orthopedic, abdominal and pelvic surgery. Antibiotic treatments, including dose and the selection of a suitable antibiotic for a particular condition or primary treatment is known to one of skill in the art (see, for example, Mullen, C. A., Pediatr Infect Dis J 12: 1138-42 (2003); Sanchez-Manuel, F. J., et al., Cochrane Database Syst Rev. 2: CD003769 (2003); van de Wetering, M. D., et al., Cochrane Database Syst Rev. 2: CD003295 (2003); Andersen B. R., et al., Cochrane Database Syst Rev. 2: CD001439 (2003); Syrjanen J., et al., Duodecim. 118: 2233-9 (2002); Esposito, S., J. Chemother. November; 13 Spec No 1(1): 12-6 (2001); Callender, D. L., Int. J. Antimicrob. Agents. August; 12 Suppl 1: S21-5, S26-7 (1999); Viscoli, C., J. Antimicrob. Chemother. June; 41 Suppl D: 65-80 (1998); Finkelstein, R., et al., Isr. J. Med. Sci. 32: 1093-7 (1996); Harbarth, S., et al., 101: 2916-21 (2000); and Jimenez, J. C., et al., Surg. Infect. (Larchmt). 4: 273-80 (2003), the teachings of all of which are hereby incorporated by reference in their entirety).

The mammal can be subject to a tissue damage consequent to the primary treatment. “Subject to a tissue damage consequent to the primary treatment,” as used herein, means that the mammal can experience an impairment in a tissue of the mammal while being exposed to or undergoing a primary treatment. The tissue damage can be a direct or indirect consequence of the primary treatment. The tissue damage consequent to the primary treatment can be deliberate tissue damage as a consequence of a primary treatment. For example, a mammal undergoing a bone marrow transplant as a primary treatment, can further undergo radiation therapy as a primary treatment. The radiation therapy is deliberately administered to the mammal to damage tissue prior to the bone marrow transplant. Agonists of bacterially-activated TLRs can be administered to the mammal while the mammal is undergoing the radiation therapy and/or the bone marrow transplant. Myeloid cells can be damaged, for example, consequent to radiation treatment in preparation for a bone marrow transplant. Administration of agonists of bacterially-activated TLRs can treat myeloid cells damaged consequent to primary treatments employed to prepare for and perform the bone marrow transplant.

In one embodiment, the agonist of bacterially-activated TLRs is administered to the mammal prior to the primary treatment. “Prior to the primary treatment,” as used herein, refers to any point in time before the mammal undergoes the primary treatment. Administration of the agonist prior to the primary treatment can prevent or minimize the tissue damage that would occur consequent to the primary treatment in the absence of the agonist. For example, the mammal can be administered the agonist before undergoing a scheduled regimen of antibiotic, chemotherapy, radiation therapy or surgical treatment, that would result in tissue damage to the mammal.

In another embodiment, the agonist of the bacterially-activated TLR is administered to the mammal following termination of the primary treatment. “Following termination of the primary treatment,” as used herein, refers to the administration of the agonist where administration of the primary treatment has ceased. For example, a mammal may have completed a prescribed treatment of chemotherapy, radiation therapy, antibiotic treatment or surgery before the mammal is administered an agonist.

In still another embodiment, the agonist of the bacterially-activated TLR is administered to the mammal concurrently with the primary treatment. “Concurrently with the primary treatment,” as used herein, refers to the administration of the agonist simultaneously with the primary treatment. Administration of the agonist concurrent with primary treatment can be administration of the agonist and the primary treatment at about the same point in time. For example, the agonist can be co-administered to the mammal with a primary treatment.

Co-administration is meant to include simultaneous or sequential administration of the agonist and primary treatment, individually or together. Where the mammal is concurrently treated with the agonist and the primary treatment, it is preferred that the administration of the agonist is conducted sufficiently close in time to the primary treatment. For example, administration of the agonist is sufficiently close in time to administration of, for example, a chemotherapeutic agent, radiation treatment, surgery, or ingestion of an antibiotic, so that the effects of the primary treatment or procedure on tissue damage, which would otherwise occur in the absence of the agonist of a bacterially-activated TLR, are absent or minimized.

The agonist can be administered to a mammal having damage to an epithelial tissue consequent to the primary treatment. In one embodiment, the epithelial tissue can be a mucosal epithelial tissue. The mucosal epithelial tissue can be a mucosal epithelial tissue of the gastro-intestinal tract (e.g., small intestine, large intestine). In another embodiment, the epithelial tissue is a skin epithelium. The keratinocytes of the epidermis and certain oral epithelium can be damaged consequent to a primary treatment. The damage to the tissue (e.g., skin epithelium) or organ (i.e., skin, the gastro-intestinal tract) can be associated with a primary treatment, such as discussed above, or by some other means, such as endogenous damage (e.g., cancer) or exogenous damage (e.g., trauma, burn, or any other inflicted wound).

The damage to the skin can also include damage to tissues other than epithelial tissue including the basement membrane of the skin and underlying stroma, including the connective tissue, extracellular matrix and cellular components (e.g., fibroblasts) of the stroma.

The agonist of a bacterially-activated TLR can be administered to a mammal having damage to at least one member selected from the group consisting of an epithelial tissue (e.g., hepatocyte), a connective tissue, a neuronal tissue and a muscle tissue consequent to the primary treatment. The connective tissue (e.g., bone marrow, blood, blood cells, stroma) can be in any organ of the body. The muscle tissue can be smooth muscle, skeletal muscle or cardiac muscle. The neuronal tissue can be neuronal tissue of the central nervous system (brain and spinal cord), the peripheral nervous system or the autonomic nervous system.

The agonist of a bacterially-activated TLR can be administered to a mammal having damage to an organ. The damage to the organ can be damage to at least one organ selected from the group consisting of the skin, heart, liver, kidney, pancreas, spleen, bone, bone marrow, pharynx and larynx.

The methods of the invention can be employed to treat inflammatory diseases in tissues and organs. For example, the methods described herein can be used to treat inflammatory bowel disease and irritable bowel syndrome.

An “effective amount,” as used herein when referring to the amount of an agonist of a bacterially-activated TLR, means that amount, or dose, of the agonist that, when administered to the mammal who is subject to a gastro-intestinal injury or tissue damage consequent to a primary treatment is sufficient for therapeutic efficacy (e.g., prevention of a gastro-intestinal injury or tissue damage consequent to a primary treatment; prevention of further gastro-intestinal injury or tissue damage consequent to a primary treatment; repair of a gastro-intestinal injury or tissue damage consequent to a primary treatment).

The methods of the present invention can be accomplished by the administration of the agonist of a bacterially-activated TLR by at least one member selected from the group consisting of oral administration or mucosal administration. Multiple routes of administration, oral and musocal can be used, including multiple forms of oral (e.g., drink, capsule) and mucosal (e.g., cream, transdermal patch) can be used to administer the agonist of the bacterially-activated TLR. Other routes of administration including intravenous and intramuscular can also be used to administer the agonist of the activated TLR (e.g., bacterially-activated TLR).

The oral administration can be by oral ingestion (e.g., drink, tablet, capsule form). Nasal administration, inhalers, suppositories, topical creams or transdermal patches can be employed for mucosal administration. Mucosal administration can be by direct application of the agonist to the mucosal surface of an organ or tissue. Mucosal administration can be by injection of an agonist into the lumen of an epithelial lined organ, for example, during a surgical procedure.

The agonists of bacterially-activated TLRs can be administered alone or as admixtures with conventional excipients, for example, pharmaceutically, or physiologically, acceptable organic, or inorganic carrier substances suitable for oral or mucosal administration that do not deleteriously react with the agonist. Suitable pharmaceutically acceptable carriers include water, salt solutions (e.g., Ringer's solution), alcohols, oils, gelatins and carbohydrates (e.g., lactose, amylose or starch, fatty acid esters, hydroxymethycellulose, and polyvinyl pyrolidine). Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances that do not deleteriously react with the agonist. The preparations can also be combined, when desired, with other active substances to reduce metabolic degradation.

More than one agonist of a bacterially-activated TLR can be administered to the mammal at one time. The agonist can be administered alone, or when combined with an admixture, in a single dose or multiple doses (in more than one dose over a period of time) to confer the desired effect (e.g., treat the gastro-intestinal injury or tissue damage consequent to a primary treatment of the mammal).

The route of administration (oral or mucosal), dosage and frequency (single or multiple doses) of the agonist of the bacterially-activated TLR administered to the mammal can vary depending upon a variety of factors, including the extent and duration of the gastro-intestinal injury, the extent and duration of the tissue damage consequent to the primary treatment, the route of administration of the agonist, the size, age, sex, health, body weight, body mass index, and diet of the mammal, kind of concurrent or primary treatment (e.g., antibiotic, chemotherapy, radiation therapy), complications from gastro-intestinal injury or tissue damage consequent to a primary treatment to the mammal or other health-related problems. Adjustment and manipulation of established dosages of the agonists of bacterially-activated TLRs (e.g., type of agonist, frequency, duration) are within the ability of those skilled in the art.

The present invention is further illustrated by way of examples, which are not intended to be limiting in any way.

### EXEMPLIFICATION

### Example 1

### TLRs Promote Tissue Repair

All complex metazoans are colonized with a myriad of microbial organisms that comprise an indigenous microflora. While present at many of the interfaces with the external world, such as the oropharynx and skin of mammals, the overwhelming majority and diversity of the endogenous bacterial flora resides at the distal alimentary tract, most notably at the colon. In the gut, over 10¹³ resident bacteria confer many benefits to intestinal physiology comprising a truly mutualistic relationship (Hooper and Gordon, 2001). The metabolism of nutrients and organic substrates, the development of intestinal epithelium, vasculature and lymphoid tissue, and the contribution to the phenomena of colonization resistance to pathogens are only a few of the ways in which the host benefits from the resident microflora present in the gut (Berg, 1996; Midvedt, 1999). However, the presence of commensal bacteria in the gut appears to be of crucial importance in the pathogenesis of human inflammatory bowel diseases (IBD), which include Crohn's disease and ulcerative colitis (Podolsky, 2002). These diseases are characterized by chronic inflammation of the intestine much of which is thought to be due to inappropriate activation of the immune system by commensal bacteria (Farrell and LaMont, 2002; Sartor, 2000).

TLRs comprise a family of pattern-recognition receptors that detect conserved molecular products of microorganisms, such as lipopolysaccharide (LPS) and lipoteichoic acid (LTA), recognized by TLR4 and TLR2, respectively (Takeda et al., 2003). TLRs function as sensors of microbial infection and are critical for the initiation of inflammatory and immune defense responses. The bacterial ligands recognized by TLRs are not unique to pathogens, but rather are shared by entire classes of bacteria, and are produced by commensal microorganisms as well. Sequestration of indigenous microflora by surface epithelia may play an important role in preventing TLR activation by commensals, whereas pathogenic bacteria are equipped with virulence factors that allow them to pass through epithelial barriers where they can be detected by TLRs expressed on macrophages and dendritic cells (Gewirtz et al., 2001; Sansonetti, 2002).

The effects of TLR ligation by commensal derived products was determined. These data show a protective role of TLRs from epithelial injury, a crucial function of TLRs in intestinal epithelial homeostasis, and provide a new perspective on host-commensal symbiosis.

Materials and Methods

Mice

MyD88−/−, TLR4−/−, TLR2−/− and WT littermates mice were bred and maintained under specific pathogen-free conditions at the animal facility of Yale University School of Medicine. These strains are maintained as F2 generations from 129/SvJ×C57BL/6.

Induction of DSS Colitis

Mice received 2% (wt/vol) DSS (40,000 kD; ICN Biochemicals), ad libitum, in their drinking water for 7 days, then switched to regular drinking water. The amount of DSS water drank per animal was recorded and no differences in intake between strains was observed. For survival studies, mice were followed for 21 days post start of DSStreatment. Mice were weighed every other day for the determination of percent weight change. This was calculated as: % weight change=(weight at day X−day 0/weight at day 0)×100. Animals were monitored clinically for rectal bleeding, diarrhea and general signs of morbidity, including hunched posture and failure to groom.

For kinetics studies, animals were sacrificed at various timepoints post the start of DSS treatment including days 0, 1, 3, 5, 7, and 9.

Radiation-Induced Injury

Mice were exposed to 10 Gy of gamma radiation at a rate of 1.8 Gy/min in a 137Cs irradiator. For survival experiments, mice were reconstituted with 3×10⁶ bone marrow cells one day post-radiation and placed on prophylactic antibiotics to control for mortality due to radiation-induced bone marrow depletion.

Scoring of Colonic Bleeding

Colonic bleeding was determined by a gross colonic blood scoring system as previously described (Siegmund et al., 2001). Colons were analyzed immediatedly after excision. Scoring was as follows: 0=lack of any gross blood visible throughout the entire colon; 1=gross blood present in <⅓ of the colon; 2=<⅔; 3=>⅔ of the colon.

Histological Scoring

Colons were excised and cut into 3 equal segments to be named proximal, middle and distal colon. Tissue was fixed with 10% neutral formalin, paraffin embedded, sectioned at 3-6 m, and stained with hematoxylin and eosin. Sections were analyzed in a blinded manner by a trained gastroentero-pathologist. Inflammatory infiltrate was scored using two different types of criteria, extent and severity of injury and character of infiltrate, where: Infiltrating Leukocyte Extent/Severity Score=Area of involved+score of severity per each layer of the intestine: mucosal, submucosal, muscularis propria, and adventitia; Inflammatory Character=Severity of Infiltrate+Area involved for each of the following: Lymphocytes, Neutrophils, Plasma cells, and Eosinophils. Epithelial Injury Score=% Area of section+Mucodepletion of Glands+Intraepithelial Lymphocytes+Ulcer/Erosion. Ulcer/Erosion Score=Ulcer/Erosion+Area involved. Histopathological changes were scored on a scale of 0-3 (where 0=none; 1=mild; 2=moderate 3=severe) for each parameter. Area involved was scored as follows: 0=no involvement; 1=<25% of section; 2=<50%; 3=<75%; 4=<100%. A score was determined for each part of the colon: proximal, middle and distal. Total scores are the sum of the scores of each individual segment.

Analyis of Red Blood Cells in Peripheral Blood

In order to determine the anemic status of experimental animals, mice were anaesthetized using metaphane and eyebled using heparin-coated capillary tubes (Fisher Scientific). Blood was transferred to microtainer tubes with K2-EDTA (Becton-Dickinson) and inverted multiple times. Red blood cell (RBC) concentration and hematocrit (percentage of whole blood in RBC) were determined by standard hematological analysis in the clinical hematology lab in the Department of Laboratory Medicine of Yale-New Haven Hospital.

Colon Organ Culture

A modification of the protocol of Siegmund et al. 2001 was used (Siegmund et al., 2001). Briefly, 1-cm segments of all three parts of the colon were washed in cold PBS supplemented with penicillin and streptomycin (Gibco). These segments were cultured in 24 well flat bottom culture plates (Falcon) in serum-free RPMI 1640 medium (Gibco) supplemented with penicillin and streptomycin. After 24 hours, supernatant fluid was collected and stored at −20° C. until analyzed.

Cytokine Measurement by Enzyme-Linked Immunosorbant Assay (ELISA)

Paired antibodies (α-mouse purified and biotinylated) and recombinant standards for TNF, IL-6, (BD Bioscience Pharmingen) and KC-1 (R&D Systems) were used to quantify factors present in supernantants of whole colon cultures. Levels in whole colon culture supernatant were standardized to the amount of total protein in supernatant by quantification by BCA analysis (Pierce) and presented as ng of cytokine per mg of protein in supernatant.

Depletion of Gut Commensal Microflora

Animals were provided ampicillin (A; 1 g/L; Sigma), vancomycin (V; 500 mg/L; Abbott Labs), neomycin sulfate (N; 1 g/L; Pharmacia/Upjohn) and metronidazole (M; 1 g/L; Sidmack Labs) in drinking water for 4 weeks prior to beginning DSS treatment and during the course of DSS administration based on a variation of the commensal depletion protocol of Fagarason et al. (Fagarasan et al., 2002). A duration of four weeks of antibiotic treatment was chosen based on both empiric bacteriologic analysis of commensal growth in feces and also to ensure that detritus of commensal bacteria which includes TLR ligands was absent from colons for one week prior to the administration of DSS. Three combinations of antibiotics were administered: For complete depletion of commensal as verified by bacteriologic analysis of colonic feces, a combination all four antibiotics was used (A/V/N/M). For selective depletions of certain classes of commensals, vancomycin and metronidazole (V/M) and neomycin sulfate and metronidazole (N/M) were used.

Bacterial Culture

For the determination of colonic microflora, fecal matter was removed from colons using sterile technique, placed in 15 ml conical tubes with thioglygolate, weighed, and vortexed until homogenous. Contents were diluted and plated on universal and differential media for the growth of anaerobes and aerobes. Colonies were counted after incubation at 37° C. for 48 hours (aerobes) and 72 hours (anaerobes). Anaerobic cultures were grown in an anaerobic chamber in the clinical microbiology lab in the Department of Laboratory Medicine of Yale-New Haven Hospital. After counting, colonies were picked and identified by biochemical analysis, morphologic appearance and Gram staining. To determine bacteremia, spleens were excised under aseptic conditions, placed in thioglycolate and made into suspension using sterile frosted glass slides. Different dilutions of these suspensions were plated, cultured aerobically and anaerobically, and analyzed as described above for fecal contents.

In Situ Intestinal Migration and Proliferation

Cells in S phase were labeled by i.p. administration of 1 mg/ml of 5′-bromo-2′-deoxyuridine (BrDU) in PBS. Intestines were excised at 2 or 24 hrs post injection and the same segment of colon (4 cm from distal end) was fixed in 10% neutral formalin buffer and embedded in paraffin. Immunohistochemistry was performed using a BrDU staining kit from BD Biosciences. Tissue were counterstained with hematoxylin. The number of cells per crypt column was quantified by counting the number of cells in intact, well oriented crypts in which adjacent nuclei and lumen were visible.

Isolation of Protein from Colonic Epithelial Cell

Epithelial cells from the large intestine of mice were isolated using the protocol of Saam and Gordon (Saam and Gordon, 1999). Protein lysates of colonic epithelial cells were made with a cocktail of protease inhibitors, quantified by BCA, and stored at −70° C.

Western Blot

Colonic protein lysates were resolved on Bis-Tris polyacrylamide gels and transferred to Immobilon paper. Blots were probed with anti-cyclin D1, c-myc (Santa Cruz), Hsp25, Hsp72 (Stressgen) and β-actin (Sigma), followed by the appropriate species specific horseradish peroxidase 2° antibody (Sigma) and developed using the ECL detection system (Amersham).

Reconstitution of Commensal-Depleted Animals with TLR Ligands

WT animals were depleted of commensals using the 4 week, A/V/N/M regimen. At week 3, drinking water was supplemented with 50 μg/μl, 10 μg/μl, or 10 ng/μl of purified E. coli 026:B6 LPS (Sigma) or 12.5 μg/μl of S. aureus LTA (Invivogen) and was continued in drinking water for the duration of DSS administration. The highest concentration of LPS (50 μg/μl) was selected to assure bioavailability of LPS at the intestinal lumen based on the oral LPS administration protocol of Tamai et al. (Tamai et al., 2002).

Statistical Analysis

Statistical analysis was performed using the paired Student's t-test. P values <0.05 were considered significant. Error bars represent ±SEM.

Results

TLR Signaling Protects from Mortality Caused by Intestinal Epithelial Injury

Current knowledge suggests that the disruption of the mucosal barrier upon injury to intestinal epithelial cells leads to the exposure of the multitude of TLR ligands produced by commensals to TLR-expressing cells, particularly macrophages, resident in the lamina propria of the intestine (Strober et al., 2002) resulting in a potent inflammatory response, intestinal inflammation, and corresponding injury. To test the effect of disrupted compartmentalization of commensals, a model of intestinal injury and inflammation was employed by the oral administration of dextran sulfate sodium (DSS), a sulfated polysaccharide known to be directly toxic to colonic epithelium (Kitajima et al., 1999).

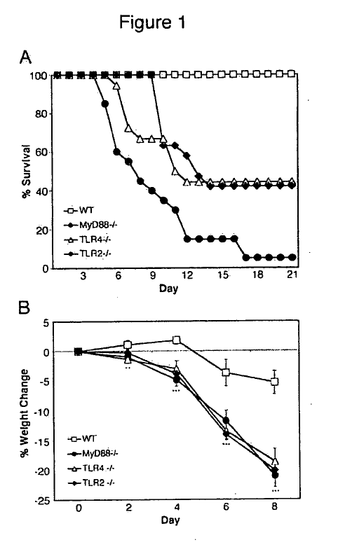

The role of TLRs in intestinal inflammation was determined in mice deficient in MyD88, an adaptor molecule essential for TLR mediated induction of inflammatory cytokines (Takeda et al., 2003), as well as mice deficient in TLR2 and TLR4. The hypothesis was that MyD88−/− mice would not be able to mount a TLR-dependent inflammatory response to commensal bacteria and, therefore, would manifest decreased intestinal pathology following DSS administration. Unexpectedly, MyD88−/− animals showed severe mortality and morbidity following the administration of DSS (2%; wt/vol) in drinking water for 7 days (FIG. 1A), unlike wildtype (WT) mice, which had 100% survival and minimal morbidity at this low dose of DSS. In accordance with the observed differences in survival, MyD88−/− mice showed more severe weight loss compared with WT controls (FIG. 1B). MyD88-dependent signaling pathway is critical for the protection against DSS-induced mortality and morbidity.

MyD88 is a signaling adaptor used by all TLRs, as well as the IL-1 receptor (IL-1R) and IL-18R (Takeda et al., 2003). Previous DSS studies using animals deficient in interleukin-1β converting enzyme (ICE), which are unable to produce IL-1β or IL-18 (Siegmund et al., 2001) and using IL-18 antagonists (Sivakumar et al., 2002) have revealed an improvement of morbidity and disease phentotype compared to WT controls. The protective role of the MyD88 signaling pathway is TLR specific and related to TLR activation, by the TLR ligands present in the colon. This conclusion was further supported by the compromised survival and severe weight loss observed in TLR2 and TLR4 deficient mice following DSS administration (FIG. 1). These results indicated that while elimination of TLR4 or TLR2 increased the susceptibility to DSS-induced disease, the severe mortality seen in MyD88−/− animals was the result of defective signaling of multiple TLRs induced by various commensal derived products.

In addition to TLRs, microbial products can be recognized by members of the NOD family of intracellular signaling proteins—NOD 1 and NOD2 (Inohara and Nunez, 2003). In particular, mutations in NOD2 have been implicated in the predisposition to Crohn's disease, although the precise role of NOD2 in the pathoetiology or pathogenesis of this condition remains unknown (Hugot et al., 2001; Ogura et al., 2001). Unlike TLRs, NODs detect their microbial ligands in the cytosol. Both NOD1 and NOD2 signal activation of NF-κB and MAP kinases through the protein kinase RIP2 (Chin et al., 2002; Kobayashi et al., 2002). Unlike MyD88 deficient mice, RIP2−/− mice are as resistant to DSS administration as wild type mice, suggesting that RIP2 dependent pathways do not play a major role in the susceptibility to colonic injury.

Severe Susceptibility to Colonic Injury in MyD88 Deficient Mice

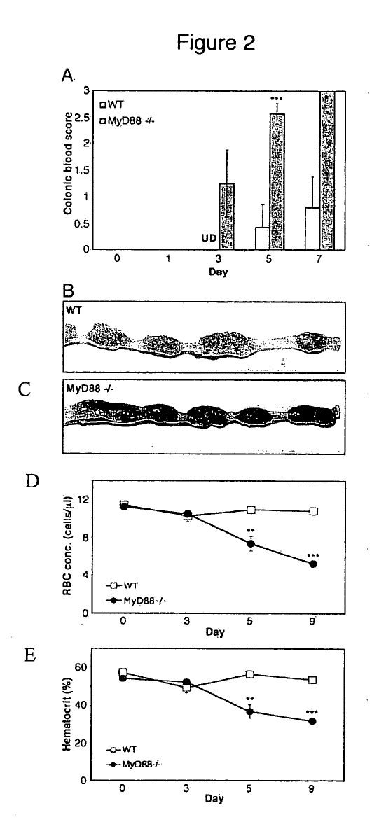

The cause of death and morbidity of the MyD88−/− animals was assessed employing several parameters. Unlike WT mice, which remained active, mobile and seemingly healthy throughout the duration of the experiment, MyD88−/− animals were moribund, with a hunched posture and defective grooming as early as day 5 post-DSS. Analysis of colons at multiple timepoints after the administration of DSS revealed bleeding in the colons of MyD88−/− mice, which occurred with more rapid onset and with much greater extent and severity compared to control animals, observed as early as day 3 and being present throughout the colon by day 5 in the MyD88−/− group (FIGS. 2A, 2B and 2C). Concordant with this increased colonic bleeding, MyD88−/− animals were severely anemic with time as determined by the measurement of red blood cells and hematocrit concentration in circulating blood (FIGS. 2D and 2E). Thus, MyD88−/− mice were dying of severe colonic bleeding and anemia upon administration of DSS. TLR2 and TLR4 deficient mice also exhibited colonic bleeding, thought not as severe as MyD88−/− animals.

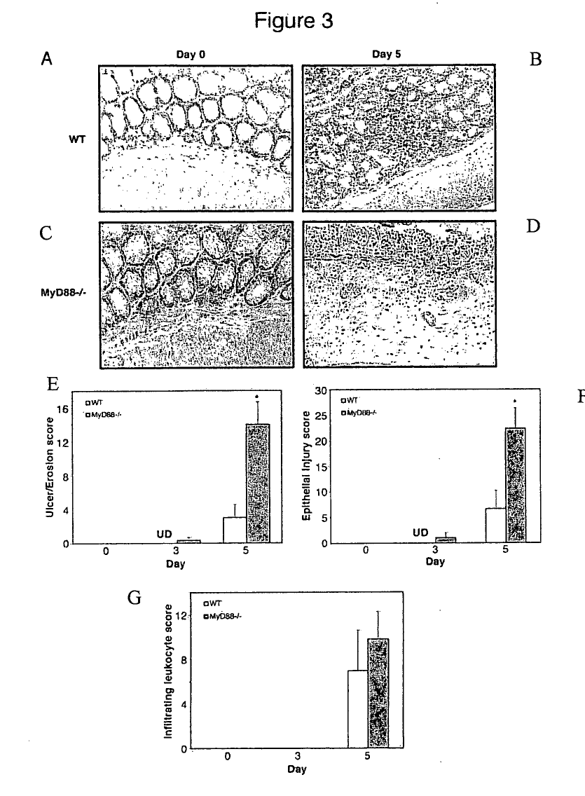

To investigate the possible mechanisms of the colonic bleeding in MyD88−/− animals, microscopic evaluation of the colons of experimental animals at multiple timepoints during the course of DSS administration was performed. Photomicrographs taken on day 5 post-DSS showed MyD88−/− colons had severe and extensive denudation of the surface epithelium (erosions) and mucodepletion of glands compared to WT control mice (FIGS. 3A-3D). Histopathological scoring of colons revealed more severe ulceration and epithelial injury at days 3 and 5 in MyD88−/− mice compared to WT controls (FIGS. 3E and 3F).

Mortality of MyD88 Deficient Mice is not Due to Commensal Overgrowth

The increased epithelial injury in colons of MyD88−/− mice, can be due to damage of uncontrolled overgrowth of commensal bacteria after disruption of the epithelial barrier, increased leukocytic infiltrate, or an inherent defect in epithelial resistance to injury and/or repair responses. The latter may be caused by a deficient induction of cytokines, cytoprotective, growth and repair factors required for protection against injury.

To determine whether the increased epithelial injury in MyD88−/− mice was due to uncontrolled overgrowth of commensural bacteria after disruption of the epithelial barrier, MyD88−/− animals were given a combination of broad-spectrum antibiotics in their drinking water for a range of 2-4 weeks prior to and during DSS administration in order to deplete the commensal flora and therefore prevent bacterial overgrowth. The sterility of the colons was confirmed by bacteriologic analysis of fecal contents (see infra). Commensal depletion of MyD88−/− animals did not prevent the morbidity or mortality seen in untreated MyD88−/− animals as these two groups of animals died with similar kinetics and hemorrhagic colons. In addition, the aerobic and anaerobic culture of spleens from MyD88−/− animals at various timepoints post-DSS administration revealed no bacteremia, showing that bacterial overgrowth was not the cause of pathology in DSS-treated MyD88−/− mice. Consistent with this conclusion, aerobic and anaerobic bacterial titers were comparable between untreated WT and MyD88−/− mice.

Mortality of MyD88 Deficient Mice is not Caused by Damage Due to Hyper-Infiltrating Leukocytes

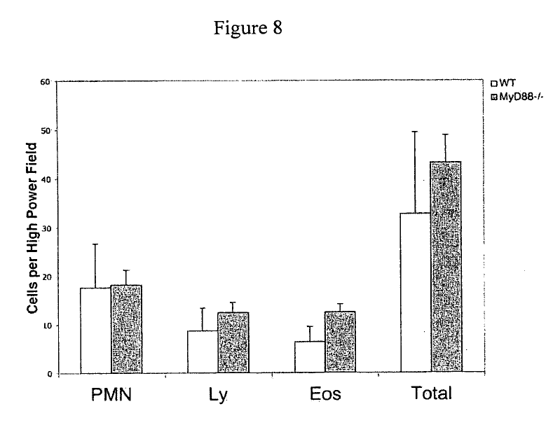

To determine whether increased epithelial injury in MyD88−/− mice was due to damage caused by infiltrating leukocytes, the leukocytic infiltrate at various timepoints post-DSS administration in WT and MyD88 deficient mice was compared. No differences in overall infiltrating leukocytes was observed (FIG. 3G). Individual analysis of infiltrating leukocytes including lymphocytes, polymorphonuclear cells, and eosinophils revealed no differences between WT and MyD88−/− colons (FIG. 8). Differences in leukocytic infiltration were not responsible for the increased colonic epithelial injury in the MyD88−/− mice. This result is consistent with findings showing that DSS-mediated pathology is independent of T, B and NK cells (Axelsson et al., 1996; Dieleman et al., 1994). This conclusion is also supported by the fact that colonic bleeding in MyD88−/− mice was apparent as early as day 3 post DSS (FIG. 2A), whereas leukocytic infiltration was not evident until day 5 post treatment (FIG. 3G).

TLR Signaling Controls Homeostasis of Intestinal Epithelium

To determine whether increased intestinal damage in MyD88−/− mice was due to a defect in the resistance of epithelial cells to direct injury several experiments were conducted. Protection from intestinal injury such as chemical, mechanical and radiation induced is determined by many factors, such as the balance of proliferation and differentiation along the crypt axis (Booth and Potten, 2001), the production of mediators involved in protecting epithelial cells from initial injury (cytoprotective) and those involved in orchestrating repair response mechanisms such as restitution (Cho and Wang, 2002; Dignass, 2001).

To determine whether the homeostatic imbalance of intestinal epithelium in the absence of TLR signaling may be responsible for the observed increased susceptibility to colonic injury, the baseline proliferative state of colonic crypts of WT and MyD88−/− mice was examined. Proliferating cells were labeled by intraperitoneal (i.p.) injection of BrDU 24 hours prior to harvesting colons.

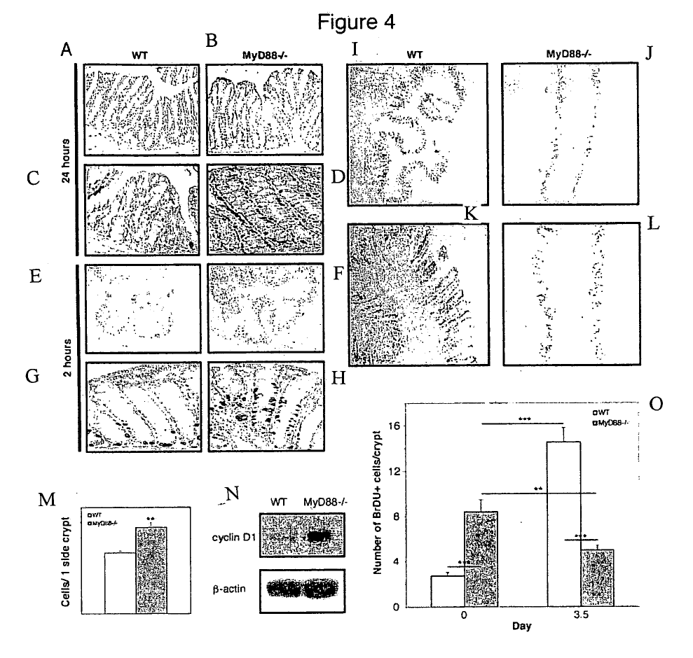

Analysis of BrDU-positive intestinal epithelial cells revealed an increased number of proliferating cells in MyD88−/− mice compared to WT controls (FIGS. 4A, 4B, 4C and 4D). The proliferating cells were clearly present in the middle and upper regions of the crypt in the MyD88−/− mice, areas of the crypt remote from the stem cell area and normally fully differentiated and non-proliferating. To exclude a possibility that BrDU-positive cells recently migrated from the stem cell area, a 2 hour BrDU labeling was determined and again showed proliferating cells at the stem cell area, and the middle and upper regions of the crypts in MyD88−/− mice (FIGS. 4E, 4F, 4G and 4H) in addition to an increase in the number of proliferating cells at baseline (FIG. 4O; day 0). Both the expanded proliferative zone and increase in number of proliferating cells in MyD88 deficient mice show dysregulated proliferation and differentiation of intestinal epithelium in the absence of TLR signals. Consistent with the higher number of proliferating cells per crypt, the average total number of cells per crypt was higher in MyD88−/− mice compared to WT controls (FIGS. 4I, 4J, 4K and 4L), and markers of cycling cells, such as cyclin D1 (FIG. 4N) and c-myc were upregulated in these cells.

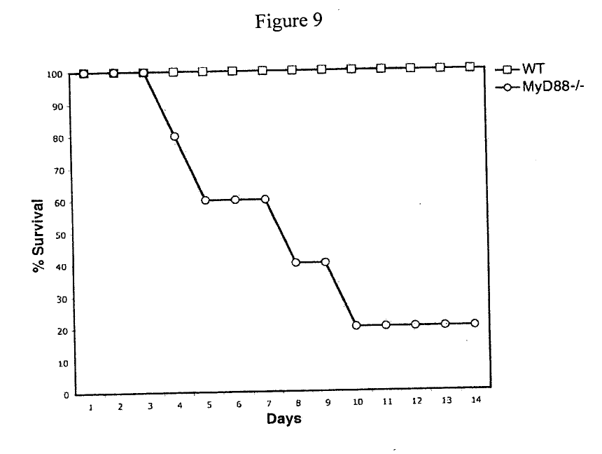

Much of the evidence for the relationship between intestinal epithelial cell cycling and susceptibility to injury comes from studies of radiation-induced injury (Neta and Okunieff, 1996) (Booth and Potten, 2001). The defect observed in intestinal epithelial homeostasis in the absence of TLR signaling should render these animals particularly susceptible to radiation-induced injury. Accordingly, MyD88−/− mice showed severe mortality upon gamma irradiation compared to WT controls (FIG. 9).

Compensatory proliferation of intestinal epithelial cells is part of the normal repair response to injury and is a reflection of the ability of cells that have survived the injurious insult to proliferate and repopulate the crypt. Crypts with increased numbers of epithelial cells in cell cycle have been shown to be more susceptible to radiation-induced injury as determined by their inability to undergo compensatory proliferation and repopulate the crypt (Booth and Potten, 2001). The susceptibility to radiation-induced injury in WT and MyD88−/− mice by comparing the compensatory proliferation and crypt repopulation in the colons of animals 3.5 days post irradiation was determined. In addition to the abnormally high level of epithelial proliferation at baseline, MyD88−/− mice sustained more severe radiation-induced epithelial damage (FIGS. 4I, 4J, 4K and 4L). These mice showed pronounced defects in crypt repopulation and compensatory proliferation compared to WT mice, as shown by the decreased number of BrDU positive cells present in the colons 3.5 days post irradiation (FIGS. 4I, 4J, 4K, 4L and 4O), and the shortened villus length compared to WT mice (FIG. 4I, 4J, 4K and 4L).

Although proliferative status is a major determinant of radiation sensitivity, compromised expression of cytoprotective and repair factors by MyD88−/− epithelium (see infra) has likely also contributed to increased susceptibility to radiation-induced injury.

TLR-Mediated Recognition of Commensals in the Colon Regulates Production of Tissue Protective Factors

In addition to homeostatic imbalance, increased susceptibility to intestinal injury may be due to defective production of cytoprotective and reparative factors. IL-6, TNF and KC-1, in addition to their role in inflammation and host defense, play direct roles in protecting various cell types, including neurons (Wang et al., 2002), hepatocytes (Bohan et al., 2003), vascular endothelium (Waxman et al., 2003), renal (Ueland et al., 2003) and lung epithelium (Ward et al., 2000), and keratinocytes (Lin et al., 2003), from injury. In the intestine, in vivo and in vitro studies have shown that these factors play beneficial roles in the response to injury through a direct effect on intestinal epithelium and the initiation of repair responses such as restitution (Yoo., 2002). In particular, IL-6 has been shown to be crucial in protecting the intestinal epithelium from injury (Tebbutt et al., 2002) possibly by regulating intestinal trefoil factor, an indispensable mediator of colonic epithelial repair (Mashimo et al., 1996). To determine whether commensals and TLRs are responsible for the induction of these cytokines for cytoprotection under normal conditions or during intestinal epithelial injury, the amounts of TNF, IL-6 and KC-1 produced by the colons of mice both at the steady-state before administration of DSS and after intestinal injury by DSS was determined.

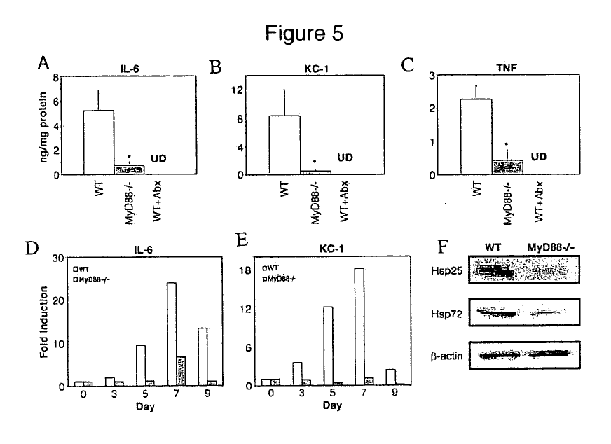

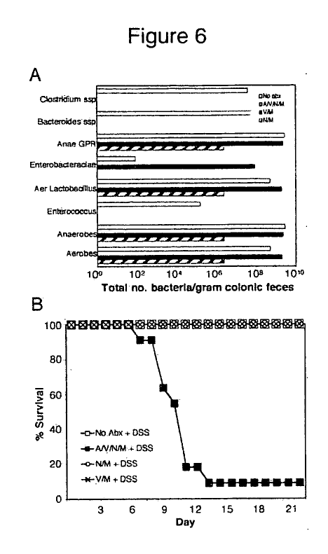

Colons of WT mice produced TNF, IL-6 and KC-1 prior to DSS administration (FIGS. 5A, 5B and 5C). The production of IL-6 and KC-1 was significantly upregulated at various time points after the administration of DSS (FIGS. 5D, 5E and 5F). In contrast, colons of MyD88−/− mice produced very low levels of TNF, IL-6 and KC-1 at the uninjured state and were not able to induce these factors after DSS administration despite severe intestinal injury (FIGS. 5A-5F). The data show that commensal bacterial products stimulate TLRs under normal conditions with intact epithelium because the production of cytokines was dependent on the presence of commensals and a functional TLR signaling pathway. Removal of commensal flora by antibiotic treatment in vivo (FIGS. 6A and 6B; see infra) completely eliminated the MyD88-dependent production of cytokines by the colon (FIGS. 5A, 5B and 5C). The effect of commensal depletion also shows that the secretion of cytokines was caused by commensal bacteria present in vivo, rather than by tissue manipulation in vitro. Although IL-6 and TNF are known to protect against intestinal injury, as shown herein, these cytokines are markers of protective responses, rather than their sole mediators. A magnitude of other cytoprotective and repair factors may be involved in TLR-mediated protection from the injury.

TLR Signaling in the Colon Controls Expression of Cytoprotective Heat Shock Proteins

In addition to growth factors and cytokines, members of the heat shock protein (hsp) families, hsp25 and hsp72, play a cytoprotective role in many cell types including intestinal epithelial cells (Malago et al., 2002). Hsps can be upregulated in vitro in intestinal epithelial cell lines by bacterial products (Kojima et al., 2003). The steady-state expression of hsp25 and hsp72 was determined in the colonic epithelium of MyD88−/− mice in vivo.

Expression of both hsp25 and hsp72 was severely diminished in the colonic epithelium of MyD88−/− mice (FIG. 5F). The cytoprotective proteins hsp25 and hsp72 may be constitutively induced by commensal products through TLRs at the steady-state. Intra-epithelial and lamina propria lymphocytes, which are known to express these hsps (Kojima et al., 2003), did not contribute to the differences in hsp expression. The numbers of different subpopulations of these cells (αβ, γδ, TCR, CD3, CD4, CD8 T cells and B cells) were comparable between WT and MyD88−/− intestines. Expression of the hsps can be induced either directly by TLR signaling in the epithelial cells, or indirectly, through the cytokines induced by TLRs. In either case, TLR signal-dependent expression of hsps 25 and 72 explains, in part, the protection from epithelial injury by TLR signaling.

Commensal Microflora is Required for the Protection from Intestinal Epithelial Injury

These data show that TLR signaling by the MyD88-dependent pathway confer protection from the mortality, morbidity, colonic bleeding and intestinal epithelial damage caused by the administration of the injurious agent DSS. These data show that commensal bacteria may be responsible for triggering TLRs and conferring protection from direct epithelial damage. To determine the role of commensal microflora in protection against colon injury, WT mice were depleted of all detectable commensals by a 4-week oral administration of vancomycin, neomycin, metronidazole and ampicillin (V/N/M/A) (FIG. 6A).

The mice that received a combination of V/N/M/A showed severe mortality and morbidity when given DSS (FIG. 6B). Consistent with commensal bacteria as responsible for the induction of protective factors by TLRs, colons of commensal-depleted animals showed no detectable levels of IL-6, TNF or KC-1 before and after administration of DSS (FIG. 5A). In contrast to mice completely devoid of detectable commensals in the gut, animals in which only certain classes of commensal bacteria were depleted through administration of selective antibiotics (FIG. 6A) showed 100% survival (FIG. 6B), with minimal morbidity and colonic bleeding similar to WT animals not given antibiotics. These data show that it is not any particular group of commensal bacteria that provides protection and commensals that are not depleted by antibiotics can still activate TLRs and induce protective and repair responses.

Recognition of Commensal Derived Ligands by TLRs is Required for the Protection from Colonic Injury

These data show that commensal bacterial products engage TLRs and confer protection against DSS-induced intestinal epithelial injury. It may be possible that the protective effect of commensal microflora was due to the beneficial effect of a particular species of commensals, through specific metabolic activity. To show that the protection was due to recognition of commensal products by TLRs (rather than due to the metabolic activity of commensals themselves), intestinal microflora-depleted animals were given either purified LPS or LTA in drinking water for 1 week prior to and during the administration of DSS. LPS and LTA would mimic the ability of gram-negative and gram-positive commensals, respectively, to trigger TLRs, but not metabolic or any other bioactivity of microflora.

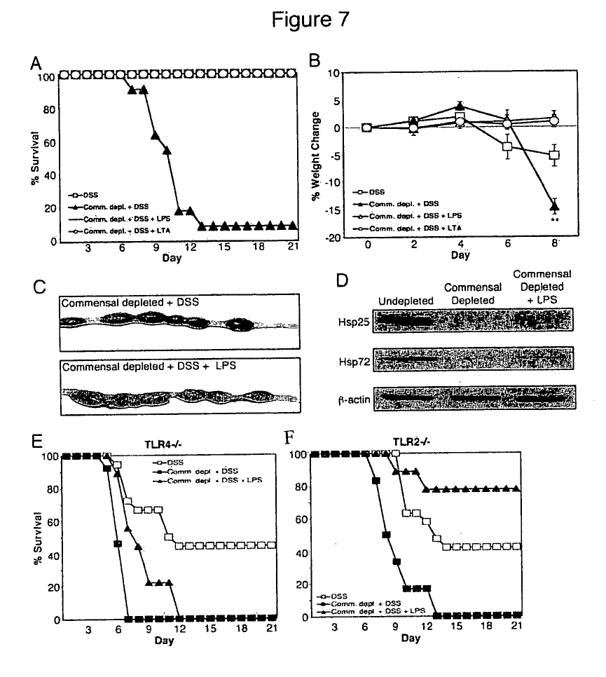

Administration of either LPS (TLR4 ligand), or LTA (TLR2 ligand) by the oral route completely protected animals from the DSS-induced mortality, morbidity and severe colonic bleeding seen in mice with colons depleted of commensal microflora (FIGS. 7A-7C). Analysis of colonic epithelium for cytoprotective heat shock proteins showed a loss of hsp 25 and 72 expression when commensals were depleted by antibiotics (FIG. 7D). This expression was upregulated upon oral administration of LPS to these commensal-depleted mice (FIG. 7D), correlating with the rescue from colonic injury upon DSS administration seen in these mice. Hsps may not the only factors responsible for LPS mediated protection, other LPS induced protective mechanisms may be involved.

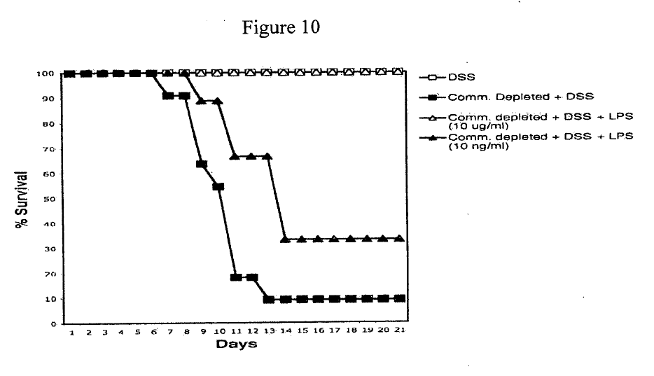

Titration of LPS in drinking water showed that the protective effect required LPS dose between 10 μg/μl (100% survival) and 10 ng/μl (30% survival) (FIG. 10). The specificity and TLR-dependence of LPS-mediated rescue was confirmed using TLR4−/− and TLR2−/− mice. Oral LPS did not rescue any of the commensal depleted TLR4 deficient mice from DSS-induced mortality, while most of the commensal depleted TLR2-deficient mice were rescued by oral LPS (FIGS. 7E and 7F). These data show that the recognition of commensal bacterial products by TLRs is responsible for the protection from mortality caused by intestinal epithelial damage.

Discussion

TLRs play a crucial role in host defense against microbial infection. The microbial ligands recognized by TLRs are not unique to pathogens and are produced by both pathogenic and commensal microorganisms. An inflammatory response to commensal bacteria may be avoided due to sequestration of microflora by surface epithelia. As shown herein, commensal bacteria are recognized by TLRs under normal steady state conditions, and this interaction plays a crucial role in the maintenance of intestinal epithelial homeostasis. Activation of TLRs by commensal microflora is critical for the protection against gut injury and associated mortality. TLRs can control intestinal epithelial homeostasis and protection from injury and show a new perspective on the evolution of host-microbial interactions.

A new role of commensal microflora and the innate immune system in mammalian physiology is described herein. In addition to the well-appreciated beneficial effects of commensals due to their metabolic activity, interaction of commensal bacterial products with host microbial pattern recognition receptors plays a critical role in resistance to epithelial injury and presumably in other aspects of epithelial homeostasis. Thus, unexpectedly, recognition of commensal bacterial products by the receptors that are critical for defense against pathogens represents a critical component of the symbiosis between the host and indigenous microflora. This interaction may be responsible for some of the other known beneficial effects of commensals on host biology.

A non-immune function of TLRs, maintenance of epithelial homeostasis and protection from direct epithelial injury, is shown herein. TLRs may directly induce the expression of several factors (in addition to heat shock proteins, IL-6, TNF and KC-1) which are involved in cytoprotection, tissue repair and angiogenesis, such as COX-2 (Rhee and Hwang, 2000), KGF-1 (Putnins et al., 2002), KGF-2 (Sanale et al., 2002), HGF (Sugiyama et al., 1996; Yoshioka et al., 2001), TGF-1 (van Tol et al., 1999), VEGF (Li et al., 2001; Zheng et al., 2002), and angiogenin-4 (Hooper et al., 2003). Many of these factors have been shown to be crucial in protecting the gut from injury (Dignass, 2001; Podolsky, 1999).

TLR mediated protection may work through two possible mechanisms that are not mutually exclusive. The first is the steady-state induction of protective factors, by the constitutive detection of lumenally derived TLR ligands on commensals by TLRs expressed on colonic epithelium. Expression of at least some TLRs, most notably TLR4, has been described in both human and mouse intestinal epithelium (Cario et al., 2002; Cario et al., 2000; Ortega-Cava et al., 2003). In vivo ablation of NF-B activation in colonic epithelium results in increased susceptibility to ischemic injury (Chen et al., 2003). Increased proliferation of colonic epithelial cells was observed in MyD88-mice (FIGS. 4A-4O). This would make them more susceptible to damage (Neta and Okunieff, 1996) (Booth and Potten, 2001). Both inflammatory cytokines and NF-κB activation have antiproliferative effects on epithelial cells (Basile et al., 2003; Booth and Potten, 2001; Neta and Okunieff, 1996; Seitz et al., 2000; Seitz et al., 1998). NF-κB participates in regulating epithelial cell turnover in the colon as p50 deficient colons have been shown to have extensive proliferative zones and elongated crypts (Inan et al., 2000), similar to what is described herein with MyD88−/− mice. The increased epithelial proliferation in MyD88−/− colons may be due, at least in part, to the disruption of TLR-induced NF-κB activation and cytokine production.

Commensal derived TLR ligands may also induce the production of protective factors upon epithelial damage. Mesenchymal-epithelial cross-talk is crucial to the orchestration of responses to tissue injury (Clark, 2003). De-sequestration of luminal repair factors from basolateral receptors is a mechanism used to detect injury and to induce a repair response in both vascular and respiratory systems. Upon disruption of the vascular endothelium, von Willebrand's factor is exposed to type IV collagen present in the basement membrane allowing for the initiation of hemostasis (de Groot, 2002). In the upper respiratory tract, epithelial damage de-sequesters heregulin, normally present on the apical side of epithelial cells, allowing its access to basolaterally located receptors and triggering them to initiate repair responses (Vermeer et al., 2003). In a similar manner, the detection of commensal derived ligands by TLRs expressed on cells resident below the basement membrane, such as fibroblasts and macrophages, may represent a signal that disruption of the epithelial barrier has occurred.

The two mechanisms are not mutually exclusive, since TLR activation on epithelial cells and myeloid cells can conceivably induce distinct tissue protection and repair responses. The data described herein show that abnormalities in intestinal epithelial homeostasis at the steady-state and a baseline defect in the production of factors such as heat shock proteins and cytokines and defects in the induced production of these factors following colonic injury, show that both epithelial detection of commensals and mesenchymal detection following damage are involved in commensal-TLR interaction required for protection. Qualitative differences and relative contributions of these two mechanisms of commensal-TLR interaction can be determined.

Inflammatory mediators have a role in host defense and wound healing (Nathan, 2002). Excessive uncontrolled inflammation results in a variety of pathological conditions, including, at the extreme, septic shock. Evolution of the inflammatory response is a result of a trade-off between its beneficial and detrimental effects. Similarly, recognition of commensal bacteria by TLRs plays an important beneficial role in the control of intestinal epithelial homeostasis and protection from direct injury, while dysregulated interaction between commensals and TLRs may promote chronic inflammation and tissue damage, such as that seen in IBD. The data described herein emphasize the existence of a crucial balance between the protective effect of TLR activation by commensals, and the detrimental effect of this interaction when it becomes dysregulated. The importance of this balance is particularly relevant for some areas of medical practice as the common clinical regimens associated with intestinal tissue damage (such as radiation, chemotherapy and colonic surgery) are universally accompanied by treatment with antibiotics to prevent opportunistic infections. The data indicate that complete depletion of microflora may have a detrimental effect on tissue repair and regeneration and that highly controlled stimulation of TLRs by their ligands (natural or synthetic) may play a beneficial role in certain situations associated with tissue damage.

Recognition of molecular patterns common to entire classes of microorganisms allows a small number of germline-encoded receptors, such as TLRs, to detect a multitude of potential microbial pathogens. The direct consequence of pattern recognition strategy is the lack of discrimination by the pattern recognition receptors between pathogenic and commensal microorganisms. It is thought that “undesired” innate immune response to commensals is normally prevented due to the sequestration of microflora by mucosal epithelial barriers, such as intestinal epithelium. The data described herein demonstrate that the ability of TLRs to recognize commensal bacterial products is not simply an unavoidable cost of pattern recognition of infection. Rather, it has its own beneficial and crucial role in mammalian physiology. Mammalian TLRs may have at least two distinct functions—protection from infection and control of tissue homeostasis (at least in the case of surface epithelia). Both functions depend on the recognition of microorganisms—pathogens and commensals, respectively. This dual function may explain why some of the TLR-induced gene products, such as inflammatory cytokines and chemokines, are involved in both host defense and tissue repair responses. One of these two TLR functions may have evolved first.

### Example 2

### TLRs in Wound Healing

Mice deficient in MyD88 (MyD88−/−; N=6) and TLRs 2 and 4 (TLR2/4−/−; N=3) and WT control (N=8) were anesthetized by intra-peritoneal injection of ketamine (100 mg/kg) and xylazine (10 mg/kg). The right flank was shaved with hand-held electronic clippers and swabbed with Betadine and 70% ethanol three times before wounding. One 4 mm punch biopsy was made in the shaved flank. At days 0, 4, 7 and 11 post-wounding, digital photos of the wound were taken along side a 4 mm-diameter paper standard for standardization of dimensions. The area of the wound at these timepoints was quantified using the National Institutes of Health (NIH) Image Version 1.61.

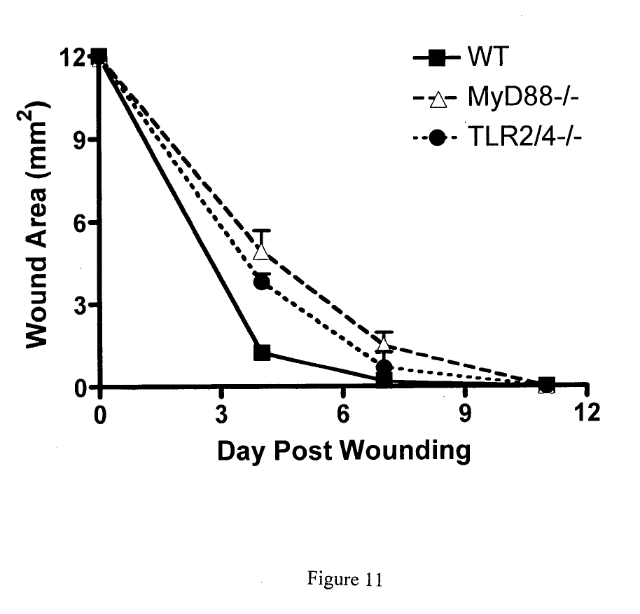

The lack of TLR signaling in MyD88−/− and TLR2/4−/− mice compared to WT mice results in delayed skin wound healing, indicating that TLR signaling promotes tissue repair and wound healing processes (FIG. 11).

### REFERENCES

Axelsson, L. G., Landstrom, E., Goldschmidt, T. J., Gronberg, A., and Bylund-Fellenius, A. C. (1996). Dextran sulfate sodium (DSS) induced experimental colitis in immunodeficient mice: effects in CD4(+)-cell depleted, athymic and NK-cell depleted SCID mice. Inflamm Res 45, 181-191.
Basile, J. R., Eichten, A., Zacny, V., and Munger, K. (2003). NF-kappaB-Mediated Induction of p21 (Cipl/Wafl) by Tumor Necrosis Factor alpha Induces Growth Arrest and Cytoprotection in Normal Human Keratinocytes. Mol Cancer Res 1, 262-270.
Berg, R. D. (1996). The indigenous gastrointestinal microflora. Trends Microbiol 4, 430-435.
Bohan, A., Chen, W. S., Denson, L. A., Held, M. A., and Boyer, J. L. (2003). TNFalpha dependent up-regulation of Lrh-1 and Mrp3(Abcc3) reduces liver injury in obstructive cholestasis. J Biol. Chem.
Booth, D., and Potten, C. S. (2001). Protection against mucosal injury by growth factors and cytokines. J Natl Cancer Inst Monogr, 16-20.
Cario, E., Brown, D., McKee, M., Lynch-Devaney, K., Gerken, G., and Podolsky, D. K. (2002). Commensal-associated molecular patterns induce selective toll-like receptor trafficking from apical membrane to cytoplasmic compartments in polarized intestinal epithelium. Am J Pathol 160, 165-173.
Cario, E., Rosenberg, I. M., Brandwein, S. L., Beck, P. L., Reinecker, H. C., and Podolsky, D. K. (2000). Lipopolysaccharide activates distinct signaling pathways in intestinal epithelial cell lines expressing Toll-like receptors. J Immunol 164, 966-972.
Chen, L. W., Egan, L., Li, Z. W., Greten, F. R., Kagnoff, M. F., and Karin, M. (2003). The two faces of IKK and NF-kappaB inhibition: prevention of systemic inflammation but increased local injury following intestinal ischemia-reperfusion. Nat Med 9, 575-581.
Chin, A. I., Dempsey, P. W., Bruhn, K., Miller, J. F., Xu, Y., and Cheng, G. (2002). Involvement of receptor-interacting protein 2 in innate and adaptive immune responses. Nature 416, 190-194.
Cho, C. H., and Wang, J. Y. (2002). Gastrointestinal mucosal repair and experimental therapeutics (Basel; N.Y., Karger). Clark, R. A. (2003). Epithelial-mesenchymal networks in wounds: a hierarchical view. J Invest Dermatol 120, ix-xi.
de Groot, P. G. (2002). The role of von Willebrand factor in platelet function. Semin Thromb Hemost 28, 133-138.
Dieleman, L. A., Ridwan, B. U., Tennyson, G. S., Beagley, K. W., Bucy, R. P., and Elson, C. O. (1994). Dextran sulfate sodium-induced colitis occurs in severe combined immunodeficient mice. Gastroenterology 107, 1643-1652.
Dignass, A. U. (2001). Mechanisms and modulation of intestinal epithelial repair. Inflamm Bowel Dis 7, 68-77.
Fagarasan, S., Muramatsu, M., Suzuki, K., Nagaoka, H., Hiai, H., and Honjo, T. (2002). Critical roles of activation-induced cytidine deaminase in the homeostasis of gut flora. Science 298, 1424-1427.
Farrell, R. J., and LaMont, J. T. (2002). Microbial factors in inflammatory bowel disease. Gastroenterol Clin North Am 31, 41-62.
Gewirtz, A. T., Navas, T. A., Lyons, S., Godowski, P. J., and Madara, J. L. (2001). Cutting edge: bacterial flagellin activates basolaterally expressed TLR5 to induce epithelial proinflammatory gene expression. J Immunol 167, 1882-1885.
Hooper, L. V., and Gordon, J. I. (2001). Commensal host-bacterial relationships in the gut. Science 292, 1115-1118.
Hooper, L. V., Stappenbeck, T. S., Hong, C. V., and Gordon, J. I. (2003). Angiogenins: a new class of microbicidal proteins involved in innate immunity. Nat Immunol 4, 269-273.
Hugot, J. P., Chamaillard, M., Zouali, H., Lesage, S., Cezard, J. P., Belaiche, J., Almer, S., Tysk, C., O'Morain, C. A., Gassull, M., et al. (2001). Association of NOD2 leucinerich repeat variants with susceptibility to Crohn's disease. Nature 411, 599-603.
Inan, M. S., Tolmacheva, V., Wang, Q. S., Rosenberg, D. W., and Giardina, C. (2000). Transcription factor NF-kappaB participates in regulation of epithelial cell turnover in the colon. Am J Physiol Gastrointest Liver Physiol 279, G1282-1291.
Inohara, N., and Nunez, G. (2003). NODs: intracellular proteins involved in inflammation and apoptosis. Nat Rev Immunol 3, 371-382.
Kitajima, S., Takuma, S., and Morimoto, M. (1999). Changes in colonic mucosal permeability in mouse colitis induced with dextran sulfate sodium. Exp Anim 48, 137-143.
Kobayashi, K., Inohara, N., Hernandez, L. D., Galan, J. E., Nunez, G., Janeway, C. A., Medzhitov, R., and Flavell, R. A. (2002). RICK/Rip2/CARDIAK mediates signalling for receptors of the innate and adaptive immune systems. Nature 416, 194-199.
Kojima, K., Musch, M. W., Ren, H., Boone, D. L., Hendrickson, B. A., Ma, A., and Chang, E. B. (2003). Enteric flora and lymphocyte-derived cytokines determine expression of heat shock proteins in mouse colonic epithelial cells. Gastroenterology 124, 1395-1407.
Li, M., Carpio, D. F., Zheng, Y., Bruzzo, P., Singh, V., Ouaaz, F., Medzhitov, R. M., and Beg, A. A. (2001). An essential role of the NF-kappa B/Toll-like receptor pathway in induction of inflammatory and tissue-repair gene expression by necrotic cells. J Immunol 166, 7128-7135.
Lin, Z. Q., Kondo, T., Ishida, Y., Takayasu, T., and Mukaida, N. (2003). Essential involvement of IL-6 in the skin wound-healing process as evidenced by delayed wound healing in IL-6-deficient mice. J Leukoc Biol 73, 713-721.
Malago, J. J., Koninkx, J. F., and van Dijk, J. E. (2002). The heat shock response and cytoprotection of the intestinal epithelium. Cell Stress Chaperones 7, 191-199.
Mashimo, H., Wu, D. C., Podolsky, D. K., and Fishman, M. C. (1996). Impaired defense of intestinal mucosa in mice lacking intestinal trefoil factor. Science 274, 262-265.
Midvedt, T. (1999). Microbial Functional Activities. In Probiotics, Other Nutritional Factors, and Intestinal Microflora, L. a. Y. Hanson, R. H., ed. (Philadelphia, Lippincott-Raven), pp. 79-96.
Nathan, C. (2002). Points of control in inflammation. Nature 420, 846-852.
Neta, R., and Okunieff, P. (1996). Cytokine-Induced Radiation Protection and Sensitization. Semin Radiat Oncol 6, 306-320.
Ogura, Y., Bonen, D. K., Inohara, N., Nicolae, D. L., Chen, F. F., Ramos, R., Britton, H., Moran, T., Karaliuskas, R., Duerr, R. H., et al. (2001). A frameshift mutation in NOD2 associated with susceptibility to Crohn's disease. Nature 411, 603-606.
Ortega-Cava, C. F., Ishihara, S., Rumi, M. A., Kawashima, K., Ishimura, N., Kazumori, H., Udagawa, J., Kadowaki, Y., and Kinoshita, Y. (2003). Strategic compartmentalization of toll-like receptor 4 in the mouse gut. J Immunol 170, 3977-3985.
Podolsky, D. K. (1999). Mucosal immunity and inflammation. V. Innate mechanisms of mucosal defense and repair: the best offense is a good defense. Am J Physiol 277, G495-499.
Podolsky, D. K. (2002). Inflammatory bowel disease. N Engl J Med 347, 417-429.
Putnins, E. E., Sanaie, A. R., Wu, Q., and Firth, J. D. (2002). Induction of keratinocyte growth factor 1 Expression by lipopolysaccharide is regulated by CD-14 and toll-like receptors 2 and 4. Infect Immun 70, 6541-6548.
Rhee, S. H., and Hwang, D. (2000). Murine TOLL-like receptor 4 confers lipopolysaccharide responsiveness as determined by activation of NF kappa B and expression of the inducible cyclooxygenase. J Biol Chem 275, 34035-34040.
Saam, J. R., and Gordon, J. I. (1999). Inducible gene knockouts in the small intestinal and colonic epithelium. J Biol Chem 274, 38071-38082.
Sanale, A. R., Firth, J. D., Uitto, V. J., and Putnins, E. E. (2002). Keratinocyte growth factor (KGF)-1 and -2 protein and gene expression in human gingival fibroblasts. J Periodontal Res 37, 66-74.
Sansonetti, P. (2002). Host-pathogen interactions: the seduction of molecular cross talk. Gut 50 Suppl 3, III2-8.
Sartor, R. B. (2000). Microbial Factor in the Pathogenesis of Crohn's Disease, Ulcerative Colitis, and Experimental Intestinal Inflammation. In Inflammatory Bowel Disease, J. B. Kirsner, ed. (Philadelphia, W.B. Saunders), pp. 153-178.
Seitz, C. S., Deng, H., Hinata, K., Lin, Q., and Khavari, P. A. (2000). Nuclear factor kappaB subunits induce epithelial cell growth arrest. Cancer Res 60, 4085-4092.
Seitz, C. S., Lin, Q., Deng, H., and Khavari, P. A. (1998). Alterations in NF-kappaB function in transgenic epithelial tissue demonstrate a growth inhibitory role for NFkappaB. Proc Natl Acad Sci USA 95, 2307-2312.
Siegmund, B., Lehr, H. A., Fantuzzi, G., and Dinarello, C. A. (2001). IL-1 beta-converting enzyme (caspase-1) in intestinal inflammation. Proc Natl Acad Sci USA 98, 13249-13254.
Sivakumar, P. V., Westrich, G. M., Kanaly, S., Garka, K., Born, T. L., Derry, J. M., and Viney, J. L. (2002). Interleukin 18 is a primary mediator of the inflammation associated with dextran sulphate sodium induced colitis: blocking interleukin attenuates intestinal damage. Gut 50, 812-820.
Strober, W., Fuss, I. J., and Blumberg, R. S. (2002). The immunology of mucosal models of inflammation. Annu Rev Immunol 20, 495-549.
Sugiyama, A., Arakaki, R., Ohnishi, T., Arakaki, N., Daikuhara, Y., and Takada, H. (1996). Lipoteichoic acid and interleukin 1 stimulate synergistically production of hepatocyte growth factor (scatter factor) in human gingival fibroblasts in culture. Infect Immun 64, 1426-1431.
Takeda, K., Kaisho, T., and Akira, S. (2003). Toll-like Receptors. Annual Review Immunology 21, 335-376.
Tamai, H., Horie, Y., Kato, S., Yokoyama, H., and Ishii, H. (2002). Long-term ethanol feeding enhances susceptibility of the liver to orally administered lipopolysaccharides in rats. Alcohol Clin Exp Res 26, 75S-80S.
Tebbutt, N. C., Giraud, A. S., Inglese, M., Jenkins, B., Waring, P., Clay, F. J., Malki, S., Alderman, B. M., Grail, D., Hollande, F., et al. (2002). Reciprocal regulation of gastrointestinal homeostasis by SHP2 and STAT-mediated trefoil gene activation in gp130 mutant mice. Nat Med 8, 1089-1097.
Ueland, J. M., Gwira, J., Liu, Z. X., and Cantley, L. G. (2003). The Chemokine KC Regulates HGF-stimulated Epithelial Cell Morphogenesis. Am J Physiol Renal Physiol.
van Tol, E. A., Holt, L., Li, F. L., Kong, F. M., Rippe, R., Yamauchi, M., Pucilowska, J., Lund, P. K., and Sartor, R. B. (1999). Bacterial cell wall polymers promote intestinal fibrosis by direct stimulation of myofibroblasts. Am J Physiol 277, G245-255.
Vermeer, P. D., Einwalter, L. A., Moninger, T. O., Rokhlina, T., Kern, J. A., Zabner, J., and Welsh, M. J. (2003). Segregation of receptor and ligand regulates activation of epithelial growth factor receptor. Nature 422, 322-326.
Wang, J., Asensio, V. C., and Campbell, I. L. (2002). Cytokines and chemokines as mediators of protection and injury in the central nervous system assessed in transgenic mice. Curr Top Microbiol Immunol 265, 23-48.
Ward, N. S., Waxman, A. B., Homer, R. J., Mantell, L. L., Einarsson, O., Du, Y., and Elias, J. A. (2000). Interleukin-6-induced protection in hyperoxic acute lung injury. Am J Respir Cell Mol Biol 22, 535-542.
Waxman, A. B., Mahboubi, K., Knickelbein, R. G., Mantell, L. L., Manzo, N., Pober, J. S., and Elias, J. A. (2003). IL-11 and IL-6 Protect Cultured Human Endothelial Cells from H2O2-Induced Cell Death. Am J Respir Cell Mol. Biol.
Yoo, J., Lotz. M. M., and Matthews, J. B. (2002). Cytokines in Restitution. In Gastrointestinal Mucosal Repair and Experimental Therapeutics, C. H. a. Y. Cho, J.-Y., ed. (Basel, Karger), pp. 14-28.
Yoshioka, T., Morimoto, Y., Iwagaki, H., Itoh, H., Saito, S., Kobayashi, N., Yagi, T., and Tanaka, N. (2001). Bacterial lipopolysaccharide induces transforming growth factor beta and hepatocyte growth factor through toll-like receptor 2 in cultured human colon cancer cells. J Int Med Res 29, 409-420.
Zheng, M., Klinman, D. M., Gierynska, M., and Rouse, B. T. (2002). DNA containing CpG motifs induces angiogenesis. Proc Natl Acad Sci USA 99, 8944-8949.

The teachings of all of the above references are hereby incorporated by reference in their entirety.

Equivalents

While this invention has been particularly shown and described with reference to preferred embodiments thereof, it will be understood that those skilled in the art that various changes in form and details may be made therein without departing from the spirit and scope of the invention as defined by the appended claims.

1. A method for treating a mammal, comprising the step of administering an agonist of a bacterially-activated TLR to a mammal subject to a gastro-intestinal injury, wherein the agonist is administered by at least one method of the group consisting of oral administration and mucosal administration and wherein the gastrointestinal injury is treated.

2. The method of claim 1, further including a second agonist that includes at least one member selected from the group consisting of a commensal bacteria and a fragment of a commensal bacteria.

3. The method of claim 2, wherein the commensal bacteria is a gastro-intestinal commensal bacteria.

4. The method of claim 1, wherein the mammal is a human.

5. The method of claim 1, wherein the agonist is administered to the mammal prior to infliction of the gastro-intestinal injury.

6. The method of claim 1, wherein the agonist is administered to the mammal subsequent to infliction of the gastro-intestinal injury.

7. The method of claim 1, wherein the agonist is administered to the mammal concurrently with infliction of the gastro-intestinal injury.

8. The method of claim 1, wherein the agonist activates at least one member selected from the group consisting of TLR2, TLR4, TLR5 and TLR6.

9. The method of claim 8, wherein the agonist of TLR5 is flagellin.

10. The method of claim 8, wherein the agonist of TLR2 is a lipoteichoic acid.

11. The method of claim 8, wherein the agonist of TLR2 is a peptidoglycan.

12. The method of claim 8, wherein the agonist of TLR4 is a lipopolysaccharide.

13. The method of claim 12, wherein the lipopolysaccharide is a lipopolysaccharide of Salmonella minnesota R595.

14. The method of claim 13, wherein the lipopolysaccharide of Salmonella minnesota R595 is monophosphoryl lipid A.

15. The method of claim 1, wherein the gastro-intestinal injury is at least one member selected from a group consisting of a small intestine injury and a large intestine injury.

16. The method of claim 1, wherein the large intestine injury is colon cancer.

17. The method of claim 1, wherein the gastro-intestinal injury is at least one member selected from the group consisting of Crohn's disease and ulcerative colitis.

18. The method of claim 1, wherein the gastro-intestinal injury is polyposis.

19. The method of claim 1, wherein the gastro-intestinal injury is injury to an epithelium of the gastro-intestinal tract.

20. The method of claim 19, wherein the epithelium is a mucosal epithelium.

21. The method of claim 20, wherein the mucosal epithelium is at least one member selected from the group consisting of a mucosal epithelial of the small intestine and mucosal epithelium of the large intestine.

22. A method for supplementing treatment of a mammal undergoing a primary treatment, wherein the mammal is subject to a tissue damage, comprising the step of administering an agonist of a bacterially-activated TLR to the mammal, wherein the agonist is administered by at least one method of the group consisting of oral administration and mucosal administration.

23. The method of claim 22, wherein the tissue damage is consequent to the primary treatment.

24. The method of claim 22, further including a second agonist that is at least one member selected from the group consisting of a commensal bacteria and a fragment of a commensal bacteria.

25. The method of claim 24, wherein the commensal bacteria is a gastrointestinal commensal bacteria.

26. The method of claim 22, wherein the mammal is a human.

27. The method of claim 22, wherein the agonist is administered to the mammal prior to the primary treatment.

28. The method of claim 22, wherein the agonist is administered to the mammal following termination of the primary treatment.

29. The method of claim 22, wherein the agonist is administered to the mammal concurrently with the primary treatment.

30. The method of claim 22, wherein the agonist is administered to a mammal undergoing as the primary treatment at least one member selected from the group consisting of a chemotherapy treatment and a radiation therapy treatment.

31. The method of claim 22, wherein the agonist is administered to a mammal undergoing surgery as the primary treatment.

32. The method of claim 22, wherein the agonist is administered to a mammal undergoing an antibiotic treatment as the primary treatment.

33. The method of claim 22, wherein the agonist is administered to a mammal undergoing a bone marrow transplant as the primary treatment.

34. The method of claim 33, wherein the mammal is further undergoing treatment with at least one member selected from the group consisting of a radiation treatment and an antibiotic treatment.

35. The method of claim 22, wherein the agonist activates at least one member selected from the group consisting of TLR2, TLR4, TLR5 and TLR6.

36. The method of claim 35, wherein the agonist of TLR5 is flagellin.

37. The method of claim 35, wherein the agonist of TLR2 is a lipoteichoic acid.

38. The method of claim 35, wherein the agonist of TLR2 is a peptidoglycan.

39. The method of claim 35, wherein the agonist of TLR4 is a lipopolysaccharide.

40. The method of claim 39, wherein the lipopolysaccharide is a lipopolysaccharide of Salmonella minnesota R595.

41. The method of claim 40, wherein the lipopolysaccharide of Salmonella minnesota R595 is monophosphoryl lipid A.

42. The method of claim 22, wherein the agonist is administered to a mammal having damage to an epithelial tissue consequent to the primary treatment.

43. The method of claim 42, wherein the epithelial tissue is a mucosal epithelial tissue.

44. The method of claim 43, wherein the mucosal epithelial tissue is a mucosal epithelial tissue of the gastro-intestinal tract.

45. The method of claim 44, wherein the gastro-intestinal tract is at least one member selected from the group consisting of the small intestine and the large intestine.

46. The method of claim 42, wherein the epithelial tissue is a skin epithelium.

47. The method of claim 22, wherein the agonist is administered to a mammal having damage to at least one member selected from the group consisting of a connective tissue, a neuronal tissue and a muscle tissue consequent to the primary treatment.
